(19) United States
(12) Patent Application Publication
(10) Pub. No.: US 2011/0152587 A1
(43) Pub. Date: Jun. 23, 2011

(54) ELECTROLUMINESCENT COMPOUNDS WITH HIGH EFFICIENCY AND ORGANIC LIGHT-EMITTING DIODE USING THE SAME

(76) Inventors: Hyo Nim Shin, Seoul (KR); Chi Sik Kim, Seoul (KR); Hyuck Joo Kwon, Seoul (KR); Young Jung Cho, Seoul (KR); Bong Ok Kim, Seoul (KR); Sung Min Kim, Seoul (KR); Seung Soo Yoon, Seoul (KR)

(21) Appl. No.: 12/936,821

(22) PCT Filed: Apr. 7, 2008
(86) PCT No.: PCT/KR2008/001951

§ 371 Date: Jan. 26, 2011

(30) Foreign Application Priority Data
Apr. 13, 2007 (KR) 10-2007-0036438

(51) Int. Cl. C07C 15/28

(52) U.S. Cl. 585/27; 585/26

(57) ABSTRACT

The present invention relates to novel organic electroluminescent compounds and an organic light-emitting diode comprising the same. The organic electroluminescent compounds according to the present invention exhibit high luminous efficiency and excellent life property as a material, so that an OLED device having very good operation life can be prepared therefrom.

### TECHNICAL FIELD

The present invention relates to novel organic electroluminescent compounds and organic light-emitting diodes using the same as electroluminescent material.

### BACKGROUND ART

Three electroluminescent materials (for red, green and blue) are employed to realize a full-colored OLED display. The important issue is to develop red, green and blue electroluminescent materials with high efficiency and long life in order to enhance the overall feature of the organic electroluminescent (EL) materials. From the aspect of function, the EL materials are classified into host materials and dopant materials. It is generally known that a device structure having the most excellent EL properties can be fabricated with an EL layer prepared by doping a dopant on a host. Recently, development of organic EL devices with high efficiency and long life comes to the fore as an urgent subject, and particularly urgent is development of a material with far better EL properties as compared to conventional EL materials as considering EL properties required for a medium to large size OLED panel. From this point of view, development of a host material is one of the most important issues to be settled. The desired properties for the host material (serving as a solid state solvent and an energy conveyer) are high purity and appropriate molecular weight to enable vapor-deposition in vacuo. In addition, glass transition temperature and thermal decomposition temperature should be high to ensure thermal stability. Further, the host material should have high electrochemical stability for providing long life. It is to be easy to form an amorphous thin film, with high adhesiveness to other adjacent materials but without interlayer migration.

Conventional host materials include diphenylvinylbiphenyl (DPVBi) from Idemitsu-Kosan and dinaphthyl-anthracene (DNA) from Kodak, but still requiring further improvements in terms of efficiency, life and color purity.

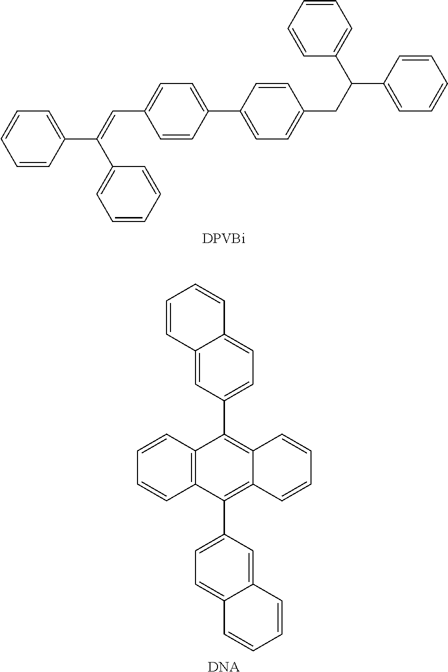

In order to develop a host material with high efficiency and long life, EL compounds based on different backbones have been disclosed, such as dispiro-fluorene-anthracene (TBSA), ter-spirofluorene (TSF) and bitriphenylene (BTP). These compounds, however, did not result in color purity and luminous efficiency at a sufficient level.

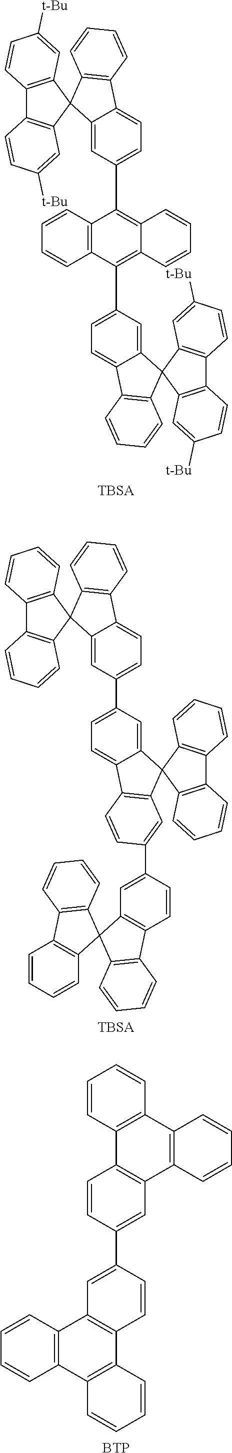

The compound TBSA as reported by Gyeongsang National University and Samsung SDI (Kwon, S. K. et al., Advanced Materials, 2001, 13, 1690; Japanese Patent Laid-Open JP 2002121547), showed luminous efficiency of 3 cd/A at 7.7 V, and relatively good color coordinate of (0.15, 0.11), but it was applied as a material for single layer, but is inappropriate for practical use. The compound TSF reported by Taiwan National University (Wu, C.-C. et al., Advanced Materials, 2004, 16, 61; US Patent Publication US 2005040392) showed relatively good external quantum efficiency of 5.3%, but it is still inappropriate for practical use. The compound BTP reported by Chingwha National University of Taiwan (Cheng, C.-H. et al., Advanced Materials, 2002, 14, 1409; US Patent Publication 2004076852) showed luminous efficiency of 2.76 cd/A and relatively good color coordinate of (0.16, 0.14), but this was still insufficient for practical use.

As described above, conventional materials are constituted of single layer, not forming host-dopant thin layer, and is difficult to be used practically from the aspect of color purity and efficiency. It lacks reliable data with respect to its long life.

In the meanwhile, according to a patent application of Mitsui Chemicals (Japan) (US Patent Publication 7,166,240), the compounds shown below have the absorption spectrum at 390 to 430 nm, with luminous efficiency of 4.6 cd/A. However, on the basis of these data, the compounds with above absorption wavelength range, electroluminescence of greenish blue color is anticipated, and the Patent Publication indicates the color as bluish green color.

Particularly, embodiment of pure blue color is impossible with the symmetrical structure of the Patent Publication, and the material, which cannot provide pure blue luminescence, is inadequate to be practically applied to a full-colored display.

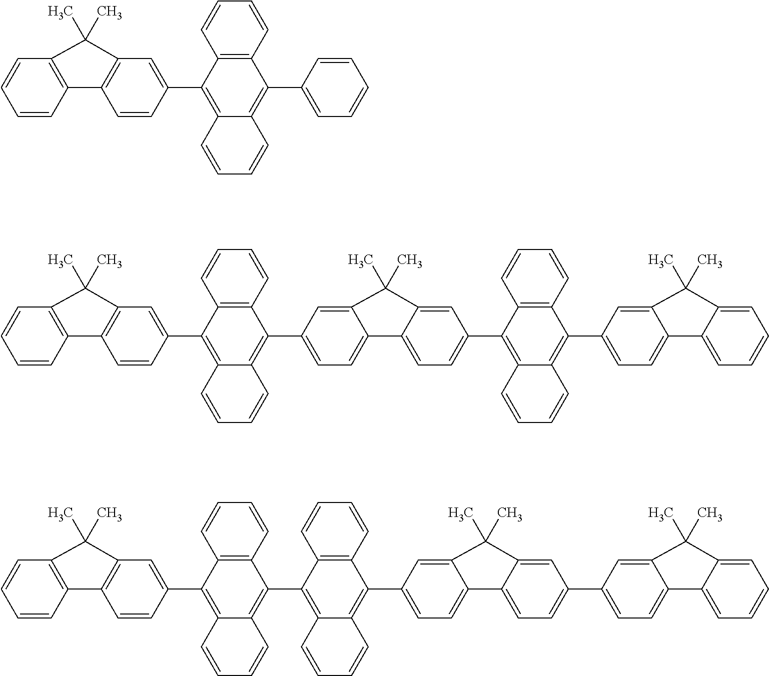

### DISCLOSURE

### Technical Problem

The object of the present invention is to provide organic EL compounds having peculiar backbone, which shows higher luminous efficiency as compared to conventional host materials, and appropriate color coordinate. Another object of the present invention is to provide organic light-emitting diodes comprising the organic EL compounds.

### Technical Solution

The present invention relates to organic EL compounds represented by Chemical Formula (1), and organic light-emitting diodes comprising the same. The organic electroluminescent compounds according to the present invention exhibit high luminous efficiency and excellent life property as a material, so that an OLED device having very good operation life can be prepared therefrom.

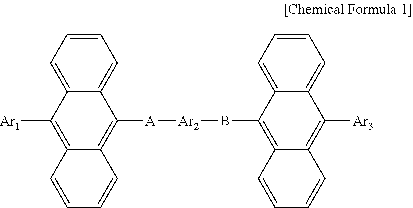

In the Chemical Formula, A and B independently represent a chemical bond or C₆-C₃₀ arylene with or without one or more substituent(s) selected from C₁-C₂₀ alkyl, C₁-C₂₀ alkoxy, C₆-C₃₀ aryl, C₄-C₃₀ heteroaryl and halogen(s), provided that A and B are not fluorenylene;

Ar₁ is an aryl group selected from the following structures, and the aryl of Ar₁ may have one substituent selected from C₁-C₂₀ alkyl, C₁-C₂₀ alkoxy, C₆-C₃₀ aryl and C₄-C₃₀ heteroaryl;

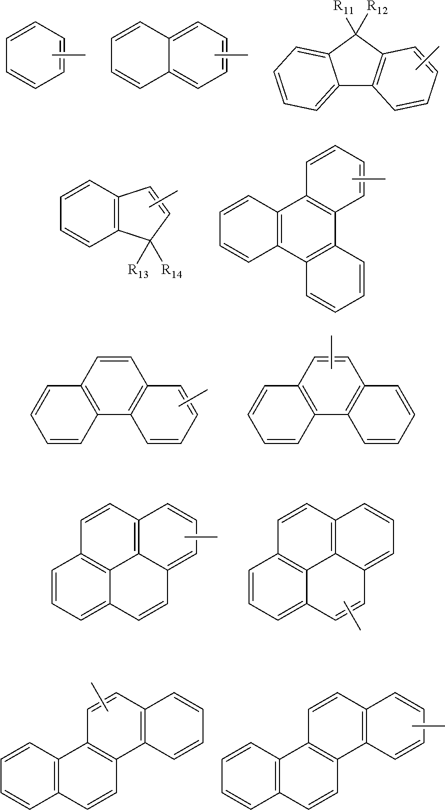

Ar₂ represents phenylene, naphthylene, anthrylene, fluorenylene, phenanthrylene, tetracenylene, naphthacenylene, chrysenylene, pentacenylene, pyrenylene, or the chemical group having the following structure; provided that Ar₂ is not fluorenylene if both A and B are chemical bonds; and the arylene of Ar₂ may have one or more substituent(s) selected from C₁-C₂₀ alkyl, C₁-C₂₀ alkoxy, C₆-C₃₀ aryl, C₄-C₃₀ heteroaryl and a halogen;

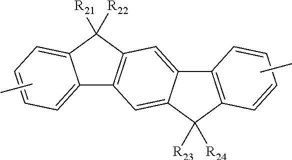

Ar₃ is an aryl group selected from the following structures, and the aryl group of Ar₃ may have one or two substituent(s) selected from C₁-C₂₀ alkyl, C₁-C₂₀ alkoxy, C₆-C₃₀ aryl and C₄-C₃₀ heteroaryl;

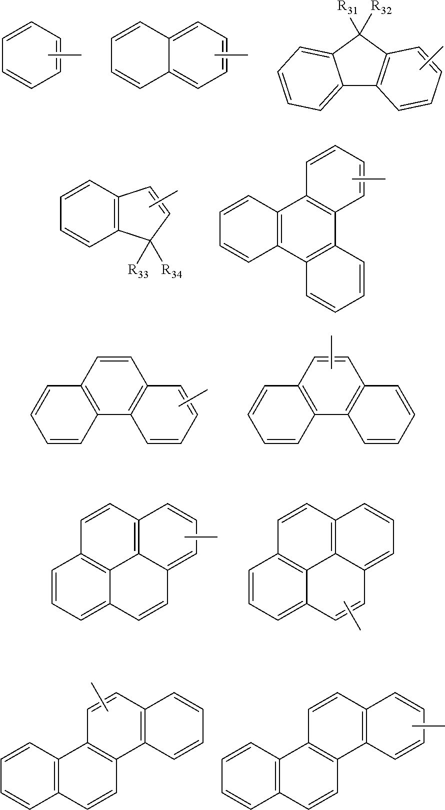

R₁₁, R₁₂, R₁₃ and R₁₄ independently represent hydrogen, C₁-C₂₀ alkyl or C₆-C₃₀ aryl;

R₂₁, R₂₂, R₂₃ and R₂₄ independently represent hydrogen, C₁-C₂₀ alkyl, C₁-C₂₀ alkoxy, C₆-C₃₀ aryl, C₄-C₃₀ heteroaryl or a halogen;

R₃₁, R₃₂, R₃₃ and R₃₄ independently represent hydrogen, C₁-C₂₀ alkyl or C₆-C₃₀ aryl.

The aryl group of Ar₁ in Chemical Formula (1) is a single ring or a fused ring, which may have one substituent selected from C₁-C₂₀ alkyl, C₁-C₂₀ alkoxy, C₆-C₃₀ aryl and C₄-C₃₀ heteroaryl. The aryl group can be specifically exemplified by the following aryl compounds, but the scope of the present invention is not restricted thereby.

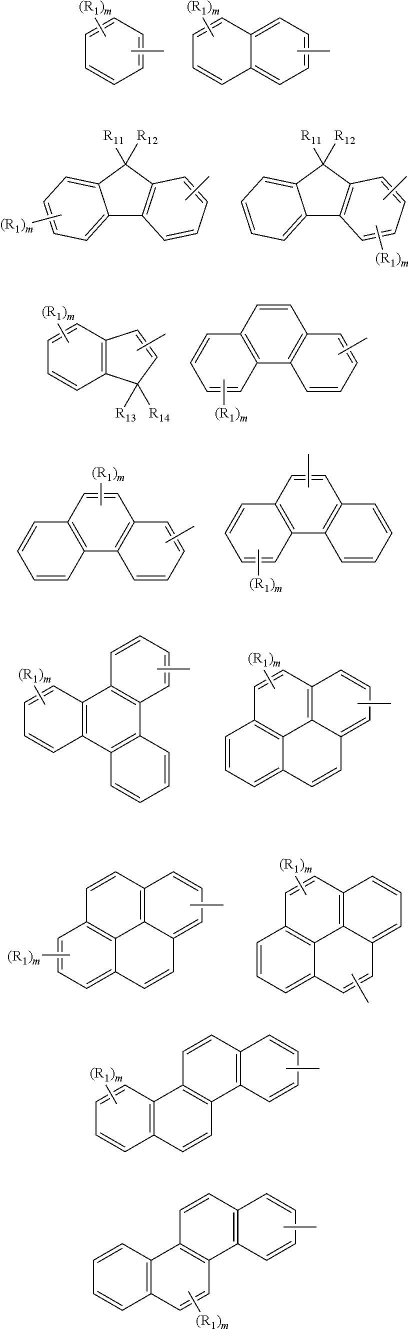

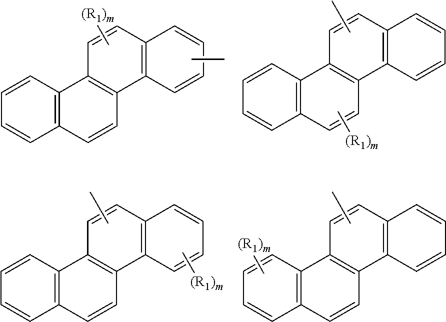

In the chemical formulas, R₁₁ and R₁₂ are defined as in Chemical Formula (1); R₁ is C₁-C₂₀ alkyl, C₁-C₂₀ alkoxy, C₆-C₃₀ aryl or C₄-C₃₀ heteroaryl; and m is an integer of 0 or 1.

The aryl group of Ar₃ is a single ring or a fused ring, and each ring may have one or two substituent(s) selected from C₁-C₂₀ alkyl, C₁-C₂₀ alkoxy, C₆-C₃₀ aryl and C₄-C₃₀ heteroaryl. The aryl group can be specifically exemplified by the following compounds, but the scope of the present invention is not restricted thereby.

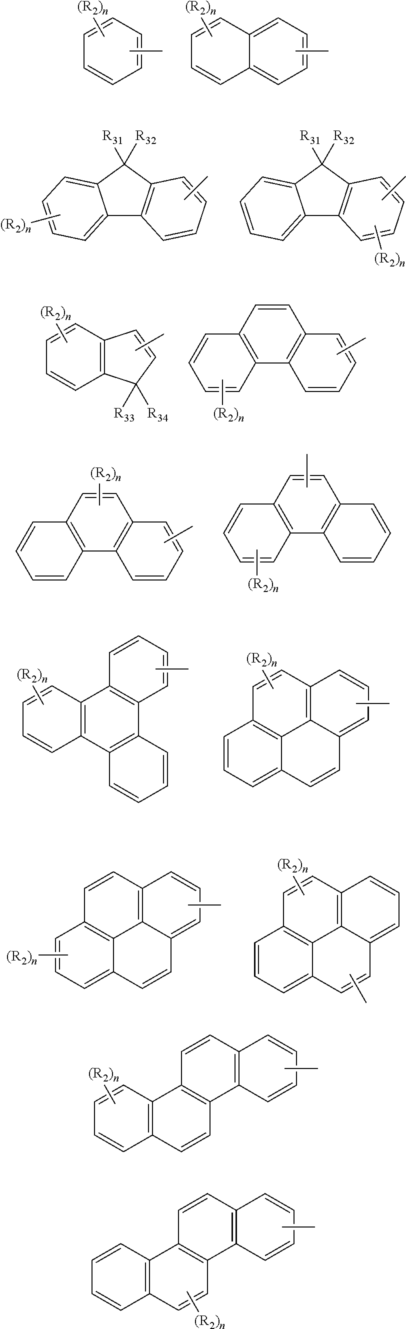

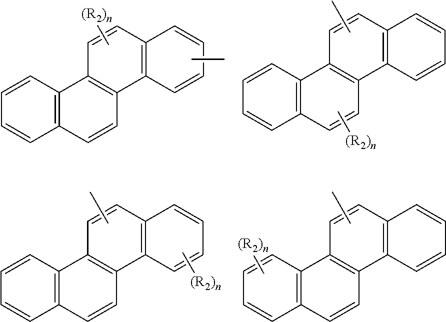

In the chemical formulas, R₃₁ and R₃₂ are defined as in Chemical Formula (1); R₂ is C₁-C₂₀ alkyl, C₁-C₂₀ alkoxy, C₆-C₃₀ aryl or C₄-C₃₀ heteroaryl; and n is an integer of 0, 1 or 2.

In the chemical formulas according to the present invention, it is referred to as “a chemical bond” if there is no element in A or B, which is simply bonded to Ar₂.

Group Ar₂ is selected from the following structures:

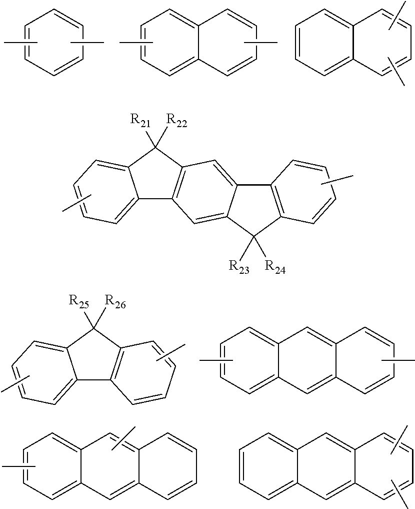

In the chemical formulas, R₂₁, R₂₂, R₂₃, R₂₄, R₂₅ and R₂₆ independently represent hydrogen, C₁-C₂₀ alkyl, C₁-C₂₀ alkoxy, C₆-C₃₀ aryl, C₄-C₃₀ heteroaryl or a halogen.

In Chemical Formula (1), -A-Ar₂—B— is selected from the following structures:

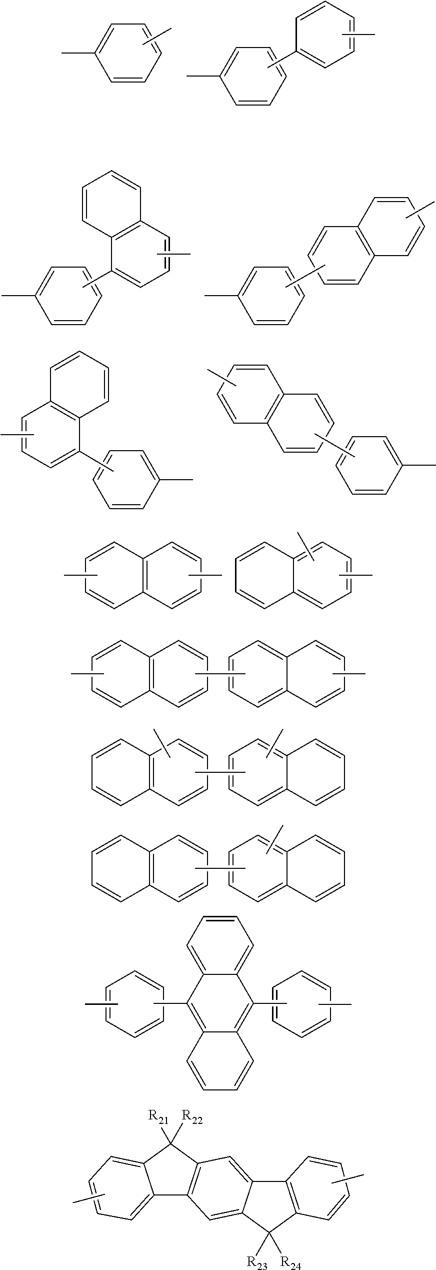

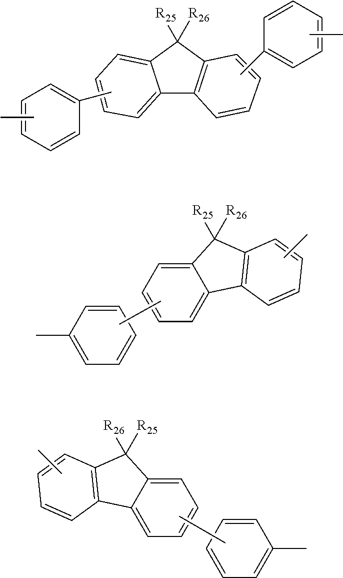

In the chemical formulas, R₂₁, R₂₂, R₂₃, R₂₄, R₂₅ and R₂₆ independently represent hydrogen, methyl, ethyl, propyl, isopropyl, butyl, isobutyl, pentyl, hexyl, ethylhexyl, heptyl, octyl, isooctyl, nonyl, decyl, dodecyl, hexadecyl, phenyl, tolyl, biphenyl, benzyl, naphthyl, anthryl or fluorenyl.

The organic electroluminescent compounds according to the present invention can be specifically exemplified by the following compounds, but the scope of the present invention is not restricted thereby.

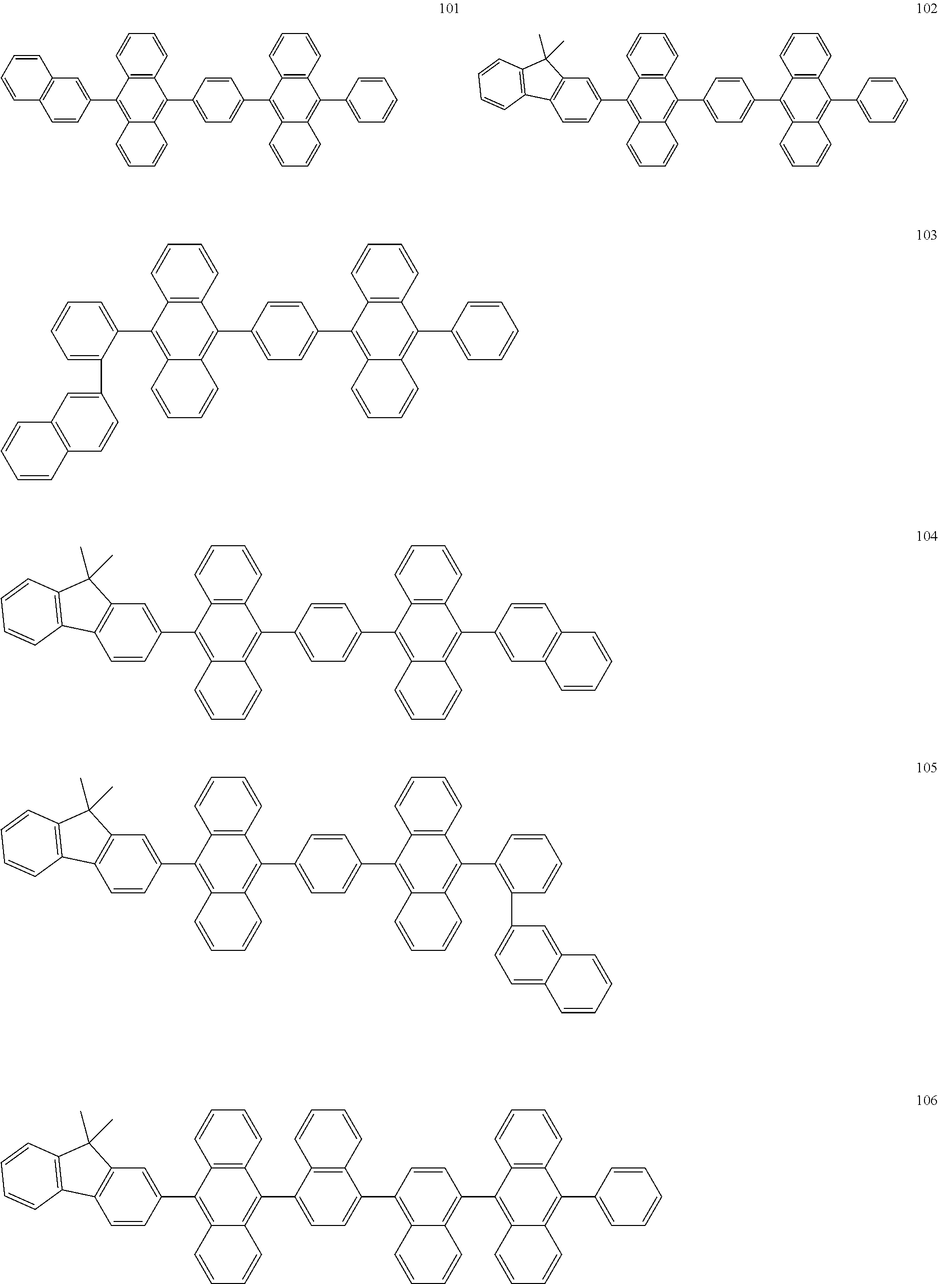

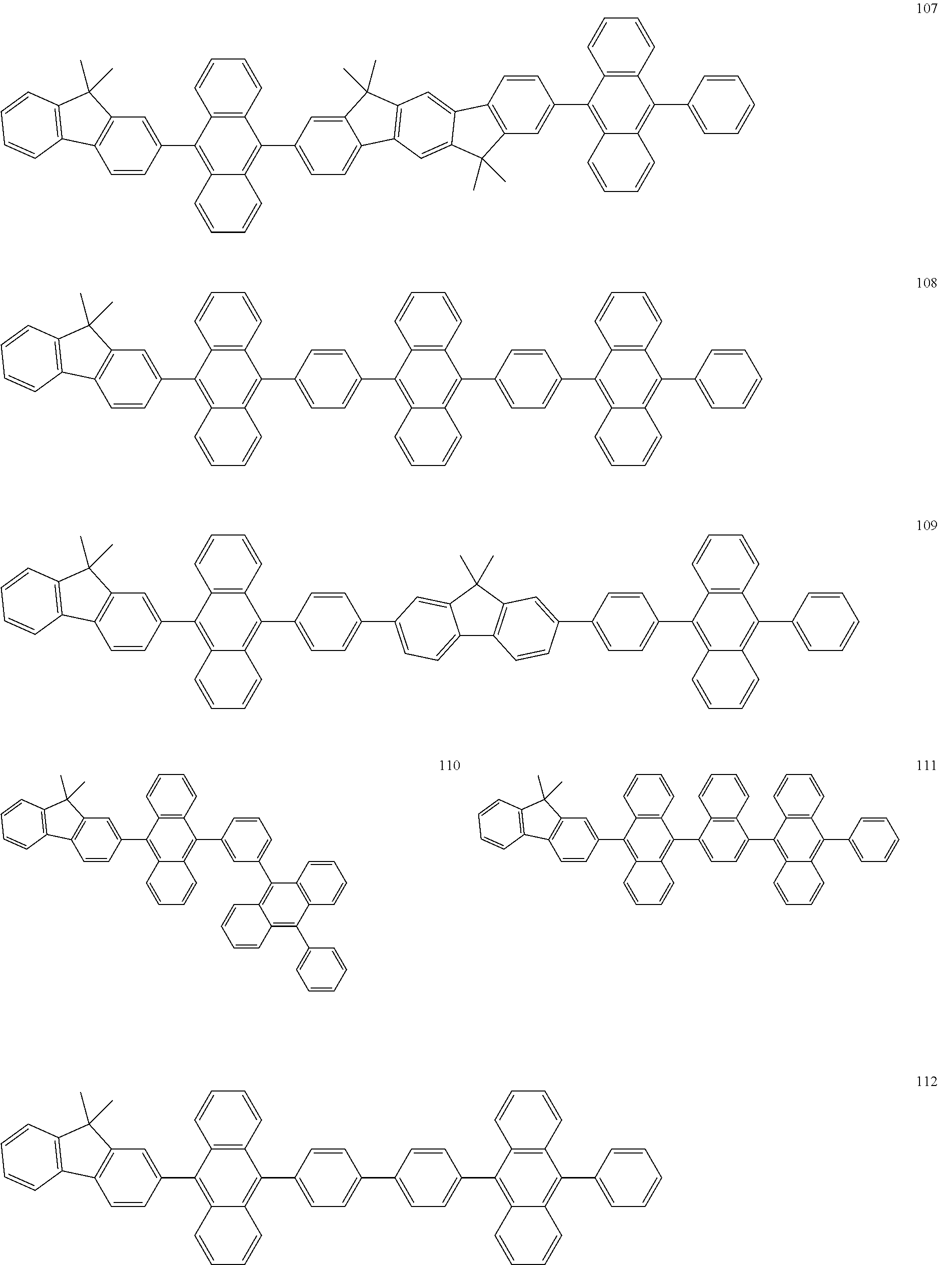

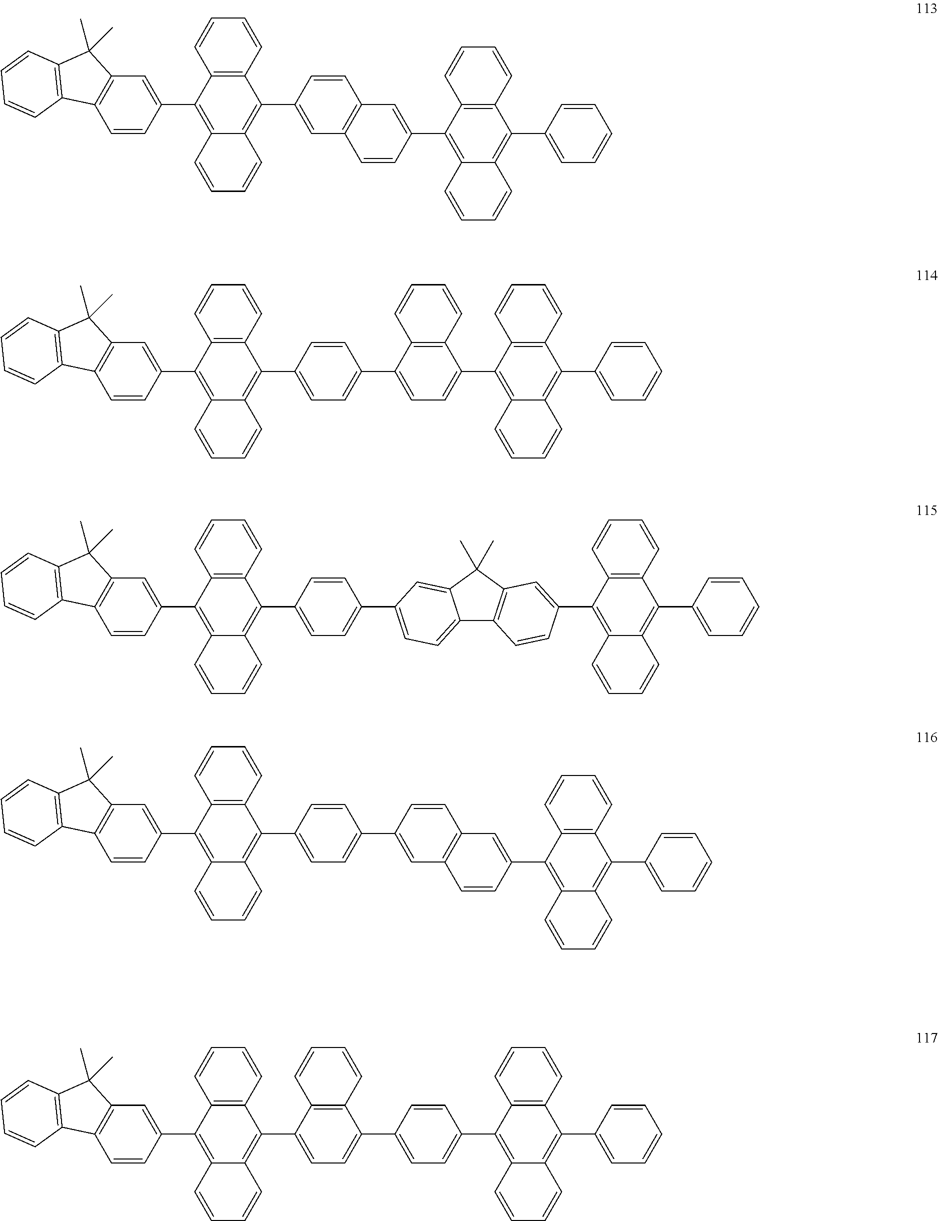

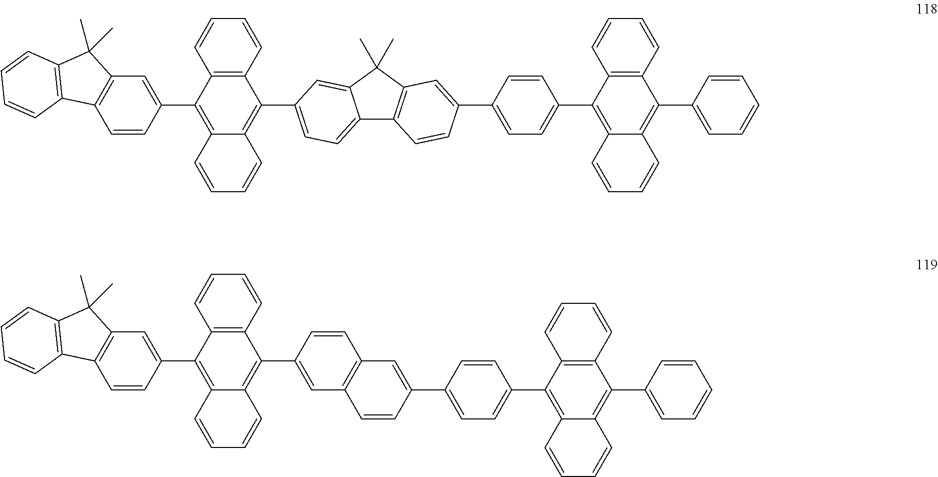

The organic light-emitting diode according to the present invention is characterized in that it employs an organic electroluminescent compound according to the present invention as a host material in the electroluminescent layer.

Basic concept of the present invention is a combination of a concept of connecting two anthracenes via a backbone having a role of a spacer with a concept of comprising asymmetric branches. This concept is a novel idea to embody increase of energy gap without shift of the electroluminescent wavelength to longer wavelength. It could be confirmed through the design, synthesis and evaluation of the molecular structure wherein the concept has been applied. In addition, it is considered as an innovative concept to exclude electrochemically brittle moiety.

The organic electroluminescent compounds according to the present invention can be prepared through a reaction route illustrated by one of Reaction Schemes (1) to (3).

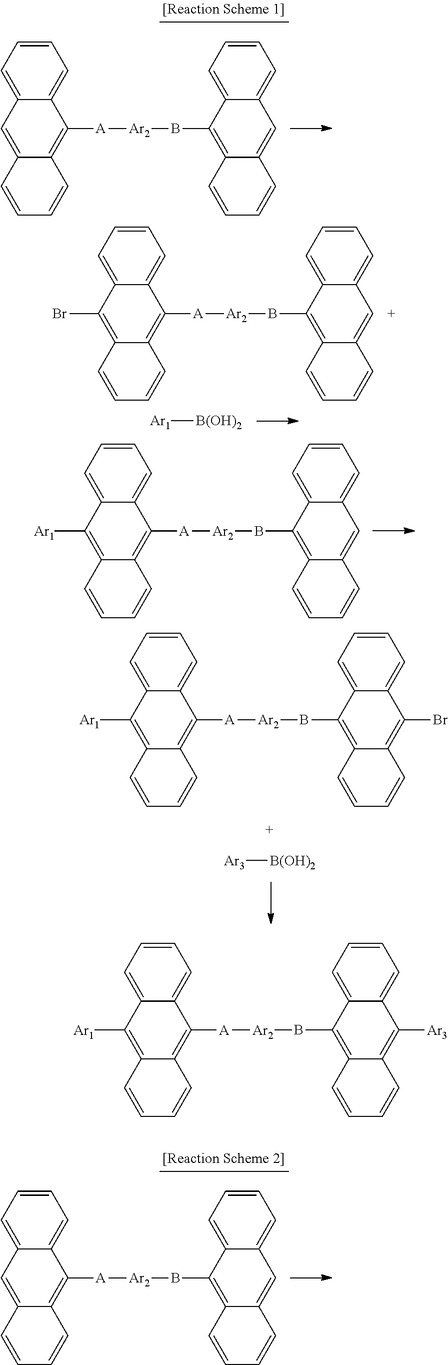

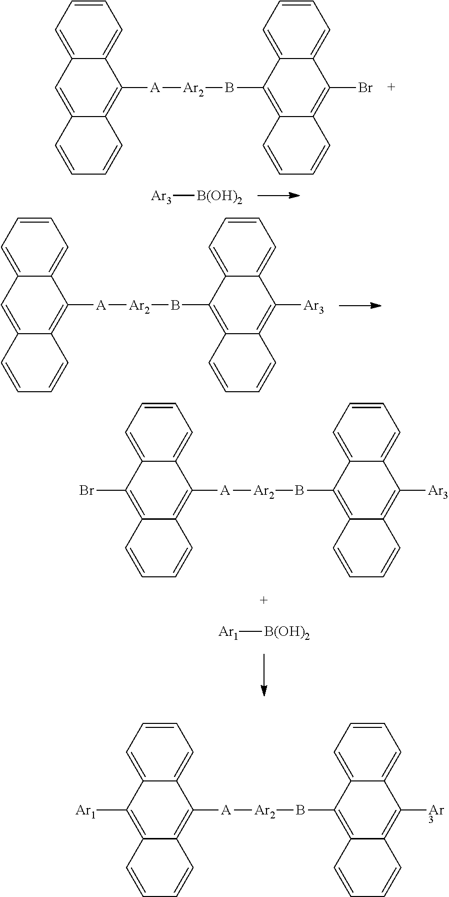

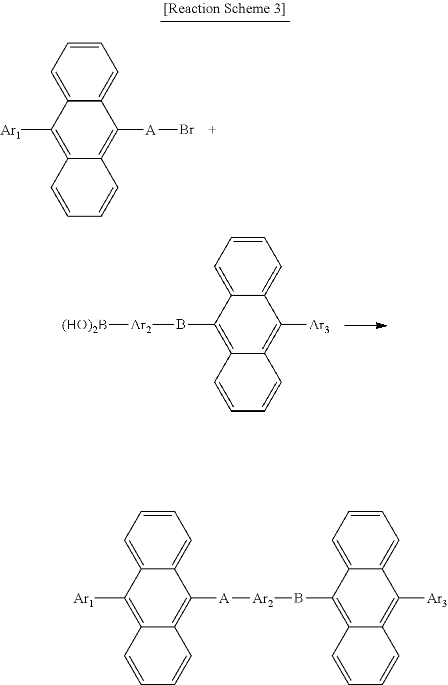

In Reaction Schemes (1) to (3), A, B, Ar₁, Ar₂ and Ar₃ is defined as in Chemical Formula (1).

### DESCRIPTION OF DRAWINGS

FIG. 1 is a cross-sectional view of an OLED;

FIG. 2 shows luminous efficiency-current density curve of Comparative Example 1 using DNA:perylene as a conventional EL material;

FIG. 3 shows current density-voltage property of a blue OLED comprising Compound (105) of the present invention as an EL material;

FIG. 4 shows luminous efficiency-current density property of a blue OLED comprising Compound (105) of the present invention as an EL material.

### DESCRIPTION OF SYMBOLS OF SIGNIFICANT PARTS OF THE DRAWINGS

1: Glass

2: Transparent electrode

3: Hole injection layer

4: Hole transportation layer

5: EL layer

6: Electron transportation layer

7: Electron injection layer

8: Al cathode

### BEST MODE

The present invention is further described with respect to the electroluminescent compounds according to the invention, a process for preparing the same and the electroluminescent properties of the device employing the same, by referring to Preparation Examples and Examples described below, which are provided for illustration only but are not intended to restrict the scope of the invention by any means.

### PREPARATION EXAMPLES

### Preparation Example 1

### Preparation of Compound (101)

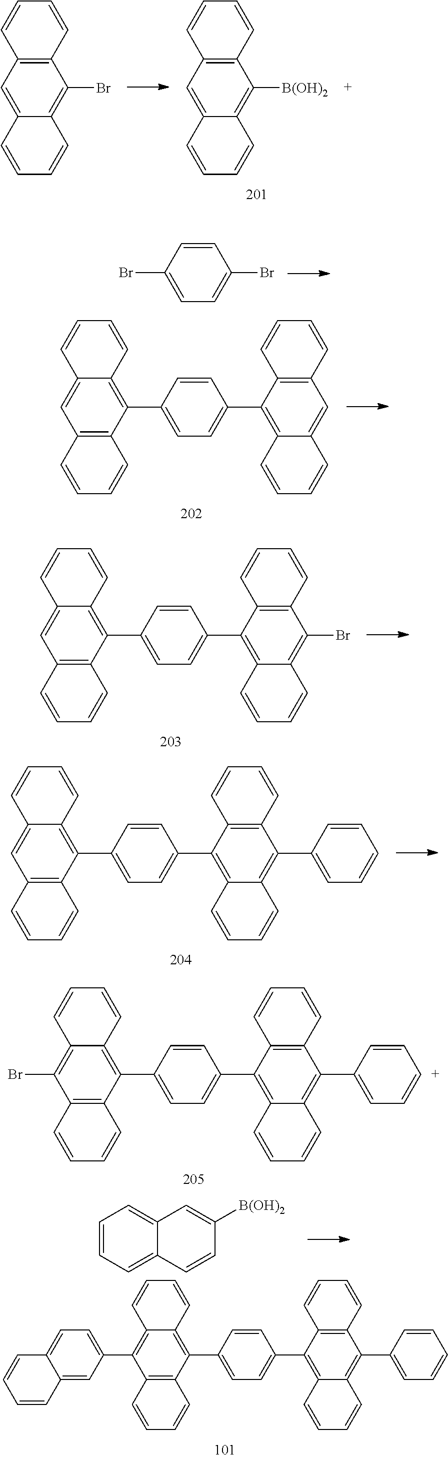

### Preparation of Compound (201)

Under nitrogen atmosphere, 9-bromoanthracene (20.0 g, 77.8 mmol) was dissolved in tetrahydrofuran (200 mL), and the solution was chilled to −78° C. To the solution, slowly added dropwise was n-butyl lithium (n-BuLi, 2.5M in hexane) (37.4 mL, 93.4 mmol). After 30 minutes, trimethylborate (17.7 mL, 155.6 mmol) was added thereto, and the resultant mixture was warmed to room temperature. After stirring at the same temperature for one day, 1N aqueous hydrochloric acid solution (200 mL) was added. The mixture was stirred for 30 minutes, and extracted from water (300 mL) and dichloromethane (200 mL). The extract was dried under reduced pressure and recrystallized from ethyl acetate (30 mL) and hexane (500 mL) to obtain the objective compound (201) (9.3 g, 41.9 mmol).

### Preparation of Compound (202)

In toluene (200 mL) and ethanol (100 mL), dissolved were Compound (201) (9.3 g, 41.9 mmol), 1,4-dibromobenzene (4.3 g, 18.2 mmol) and tetrakispalladium (O) triphenylphosphine (Pd(PPh₃)₄) (2.1 g, 1.8 mmol). To the solution, aqueous 2M sodium carbonate solution (100 mL) was added, and the mixture was stirred at 120° C. under reflux for 12 hours. Then, the mixture was cooled to 25° C., and the reaction was quenched by adding distilled water (100 mL). The reaction mixture was extracted with ethyl acetate (100 mL), and the extract was dried under reduced pressure. Recrystallization from tetrahydrofuran (20 mL) and methanol (300 mL) gave the objective compound (202) (6.5 g, 15.1 mmol).

### Preparation of Compound (203)

Under nitrogen atmosphere, Compound (202) (6.5 g, 15.1 mmol) and N-bromosuccinimide (3.0 g, 16.6 mmol) were dissolved in dichloromethane (200 mL), and the solution was stirred at 25° C. for one day. Then, the reaction was quenched by adding distilled water (200 mL), and the mixture was extracted with dichloromethane (100 mL). The extract was dried under reduced pressure and recrystallized from tetrahydrofuran (20 mL) and methanol (200 mL) to obtain the objective compound (203) (6.9 g, 13.5 mmol).

### Preparation of Compound (204)

In toluene (150 mL) and ethanol (70 mL), dissolved were Compound (203) (6.9 g, 13.5 mmol), phenylboronic acid (2.0 g, 16.2 mmol) and tetrakispalladium (O) triphenylphosphine (Pd(PPh₃)₄) (1.6 g, 1.4 mmol). Aqueous 2M sodium carbonate solution (70 mL) was added to the solution, and the mixture was stirred at 120° C. under reflux for 6 hours. Then, the temperature was lowered to 25° C., and the reaction was quenched by adding distilled water (100 mL). The reaction mixture was extracted with ethyl acetate (100 mL), and the extract was dried under reduced pressure. Recrystallization from tetrahydrofuran (20 mL) and methanol (200 mL) gave the objective compound (204) (5.8 g, 11.4 mmol).

### Preparation of Compound (205)

Under nitrogen atmosphere, Compound (204) (5.8 g, 11.4 mmol), N-bromosuccinimide (2.4 g, 13.7 mmol) were dissolved in dichloromethane (150 mL), and the solution was stirred at 25° C. for one day. The reaction was then quenched by adding distilled water (150 mL), and the reaction mixture was extracted with dichloromethane (100 mL). The extract was dried under reduced pressure, and recrystallized from tetrahydrofuran (20 mL) and methanol (200 mL) to obtain the objective compound (205) (5.6 g, 9.6 mmol).

### Preparation of Compound (101)

In toluene (100 mL) and ethanol (50 mL), dissolved were Compound (205) (5.6 g, 9.6 mmol), 2-naphthylboronic acid (2.0 g, 11.5 mmol) and tetrakispalladium (O) triphenylphosphine (Pd(PPh₃)₄) (1.2 g, 1.0 mmol). Aqueous 2M sodium carbonate solution (50 mL) was added to the solution, and the mixture was stirred at 120° C. under reflux for 6 hours. Then, the temperature was lowered to 25° C., and the reaction was quenched by adding distilled water (100 mL). The reaction mixture was extracted with ethyl acetate (100 mL), and the extract was dried under reduced pressure. Recrystallization from tetrahydrofuran (20 mL) and methanol (200 mL) gave the objective compound (101) (4.5 g, 7.1 mmol, 74%).

¹H NMR(CDCl₃, 200 MHz) δ=7.22(m, 1H), 7.32-7.34(m, 12H), 7.48-7.54(m, 7H), 7.67-7.73(m, 11H), 7.89(d, 1H)

MS/FAB: 633.8(found) 632.8(calculated)

### Preparation Example 2

### Preparation of Compound (102)

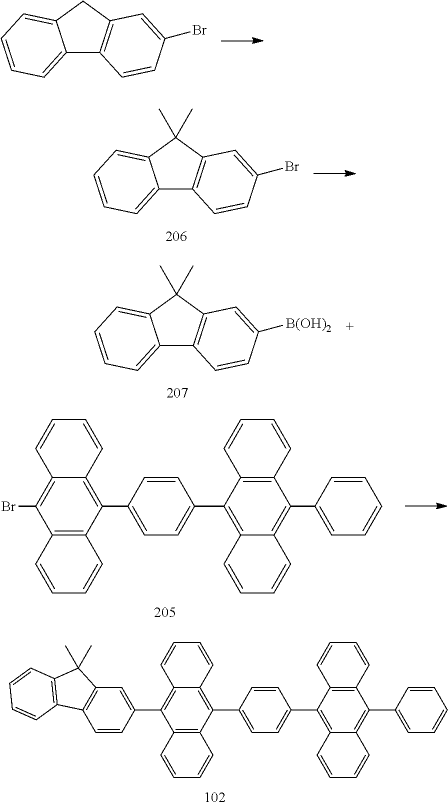

### Preparation of Compound (206)

In dimethylsulfoxide (150 mL), dissolved were 2-bromofluorene (15.0 g, 61.0 mmol), potassium iodide (KI) (1.0 g, 0.6 mmol) and potassium hydroxide (15.5 g, 270.0 mmol). Iodomethane (8.7 mL, 139.0 mmol) was added thereto at 10° C., and the temperature was raised to 30° C. After stirring the mixture at the same temperature for 12 hours, the reaction mixture was poured to distilled water (500 mL), and stirred for 30 minutes. The solid produced was filtered, and washed once with hexane to obtain the objective compound (206) (14.2 g, 52.0 mmol).

### Preparation of Compound (207)

Under nitrogen atmosphere, Compound (206) (14.2 g, 52.0 mmol) was dissolved in tetrahydrofuran (200 mL), and the solution was chilled to −78° C. To the solution, slowly added dropwise was n-butyl lithium (n-BuLi, 2.5M in hexane) (25.0 mL, 62.4 mmol). After 30 minutes, trimethylborate (11.8 mL, 104.0 mmol) was added dropwise thereto, and the resultant mixture was warmed to room temperature. After stirring at the same temperature for one day, 1N aqueous hydrochloric acid solution (100 mL) was added. The mixture was stirred for 30 minutes, and extracted with water (200 mL) and dichloromethane (400 mL). The extract was dried under reduced pressure and recrystallized from ethyl acetate (20 mL) and hexane (300 mL) to obtain the objective compound (207) (7.8 g, 32.8 mmol).

### Preparation of Compound (102)

In toluene (400 mL) and ethanol (200 mL), dissolved were Compound (207) (7.8 g, 32.8 mmol), Compound (205) (16.0 g, 27.3 mmol) and tetrakispalladium (O) triphenylphosphine (Pd(PPh₃)₄) (3.1 g, 2.7 mmol). To the solution, aqueous 2M sodium carbonate solution (150 mL) was added, and the mixture was stirred at 120° C. under reflux for 6 hours. Then, the mixture was cooled to 25° C., and the reaction was quenched by adding distilled water (300 mL). The reaction mixture was extracted with ethyl acetate (200 mL), and the extract was dried under reduced pressure. Recrystallization from tetrahydrofuran (40 mL) and methanol (400 mL) gave the objective compound (102) (12.7 g, 18.2 mmol, 67%).

¹H NMR(CDCl₃, 200 MHz) δ=1.67(s, 6H), 7.22-7.32(m, 13H), 7.48-7.67(m, 16H), 7.84-7.90(m, 2H)

MS/FAB: 699.3(found) 698.3(calculated)

### Preparation Example 3

### Preparation of Compound (103)

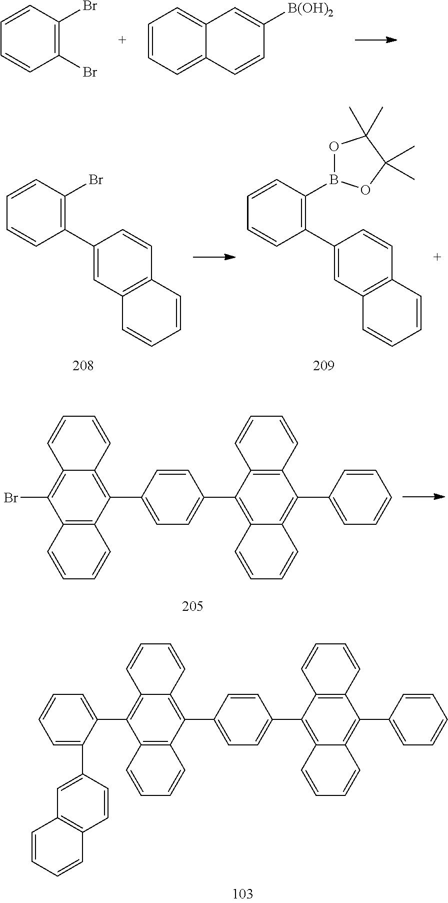

### Preparation of Compound (208)

In toluene (400 mL) and ethanol (200 mL), dissolved were 1,2-dibromobenzene (10.0 g, 42.4 mmol), 2-naphthylboronic acid (8.7 g, 50.9 mmol) and tetrakispalladium (O) triphenylphosphine (Pd(PPh₃)₄) (4.9 g, 4.2 mmol). To the solution, aqueous 2M sodium carbonate solution (200 mL) was added, and the mixture was stirred at 120° C. under reflux for 3 hours. Then, the mixture was cooled to 25° C., and the reaction was quenched by adding distilled water (300 mL). The reaction mixture was extracted with ethyl acetate (200 mL), and the extract was dried under reduced pressure. Purification via column chromatography (hexane) gave the objective compound (208) (8.4 g, 29.7 mmol).

### Preparation of Compound (209)

Compound (208) (8.4 g, 29.7 mmol) was dissolved in thoroughly purified tetrahydrofuran (200 mL) under nitrogen atmosphere, and the solution was chilled to −78° C. To the solution, n-butyl lithium (1.6M in hexane) (22.3 mL, 35.6 mmol) was slowly added dropwise, and the mixture was stirred for 30 minutes. Then, 2-isopropoxy-4,4,5,5-tetramethyl-1,3,2-dioxaborolane (12.1 mL, 59.4 mmol) was added thereto. The temperature was slowly raised and stirred at 25° C. for one day, and distilled water (300 mL) was added thereto to quench the reaction. The reaction mixture was extracted with ethyl acetate (200 mL), and the extract was dried under reduced pressure. Recrystallization from tetrahydrofuran (20 mL) and methanol (200 mL) gave the objective compound (209) (6.2 g, 18.8 mmol).

### Preparation of Compound (103)

In toluene (200 mL), dissolved were Compound (209) (6.2 g, 18.8 mmol), Compound (105) (9.2 g, 15.7 mmol), tetrakispalladium (O) triphenylphosphine (Pd(PPh₃)₄) (1.8 g, 1.6 mmol) and Aliquat 336 (0.8 mL, 1.6 mmol). To the solution, aqueous 2M sodium carbonate solution (80 mL) was added, and the mixture was stirred at 120° C. under reflux for 6 hours. Then, the mixture was cooled to 25° C., and the reaction was quenched by adding distilled water (300 mL). The reaction mixture was extracted with ethyl acetate (100 mL), and the extract was dried under reduced pressure. Recrystallization from tetrahydrofuran (30 mL) and methanol (200 mL) gave the objective compound (103) (7.6 g, 10.7 mmol, 69%).

¹H NMR(CDCl₃, 200 MHz) δ=7.22-7.34 (m, 15H), 7.48-7.54(m, 9H), 7.68-7.73(m, 11H), 7.89(d, 1H)

MS/FAB: 709.2(found) 708.2(calculated)

### Preparation Example 4

### Preparation of Compound (104)

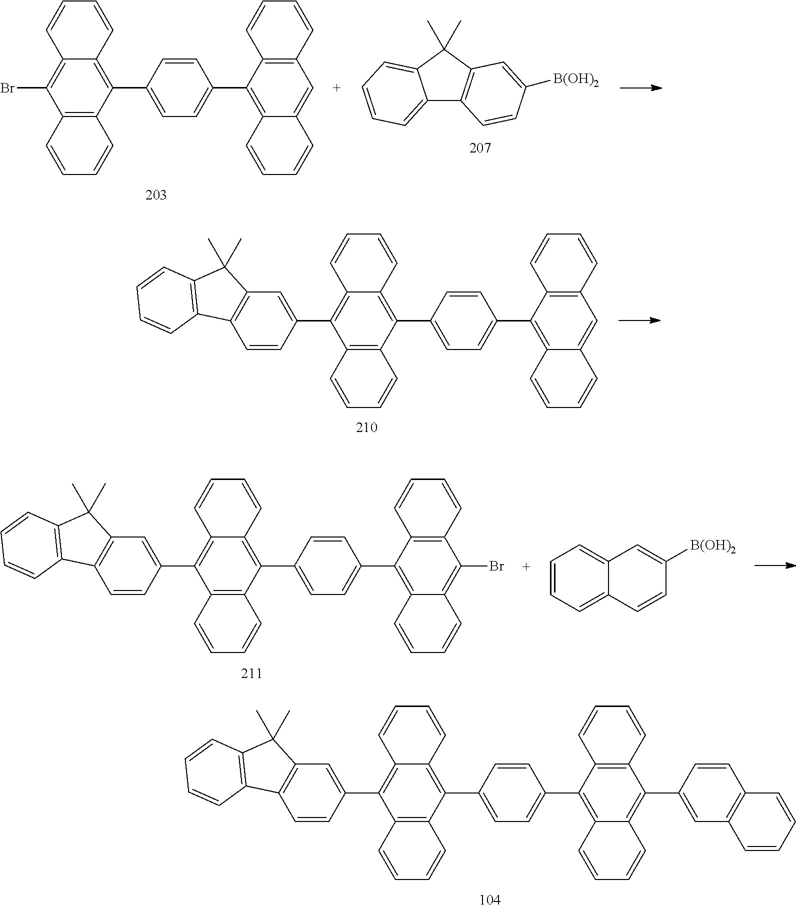

### Preparation of Compound (210)

In toluene (200 mL) and ethanol (100 mL), dissolved were Compound (203) (10.0 g, 19.6 mmol), Compound (207) (5.6 g, 23.6 mmol) and tetrakispalladium (O) triphenylphosphine (Pd(PPh₃)₄) (2.3 g, 2.0 mmol). To the solution, aqueous 2M sodium carbonate solution (100 mL) was added, and the mixture was stirred at 120° C. under reflux for 5 hours. Then, the mixture was cooled to 25° C., and the reaction was quenched by adding distilled water (300 mL). The reaction mixture was extracted with ethyl acetate (200 mL), and the extract was dried under reduced pressure. Recrystallization from tetrahydrofuran (30 mL) and methanol (200 mL) gave the objective compound (210) (8.5 g, 13.6 mmol).

### Preparation of Compound (211)

Compound (210) (8.5 g, 13.6 mmol) obtained as above and N-bromosuccinimide (2.9 g, 16.3 mmol) were dissolved in dichloromethane (200 mL) under nitrogen atmosphere, and the solution was stirred at 25° C. for one day. Then, distilled water (200 mL) was added to quench the reaction. The reaction mixture was extracted with dichloromethane (100 mL), and the extract was dried under reduced pressure. Recrystallization from tetrahydrofuran (20 mL) and methanol (200 mL) gave the objective compound (211) (7.6 g, 10.8 mmol).

### Preparation of Compound (104)

In toluene (100 mL) and ethanol (50 mL), dissolved were Compound (211) (7.6 g, 10.8 mmol), 2-naphthylboronic acid (2.2 g, 13.0 mmol) and tetrakispalladium (O) triphenylphosphine (Pd(PPh₃)₄) (1.3 g, 1.1 mmol). To the solution, aqueous 2M sodium carbonate solution (54 mL) was added, and the mixture was stirred at 120° C. under reflux for 6 hours. Then, the mixture was cooled to 25° C., and the reaction was quenched by adding distilled water (200 mL). The reaction mixture was extracted with ethyl acetate (100 mL), and the extract was dried under reduced pressure. Recrystallization from tetrahydrofuran (10 mL) and methanol (100 mL) gave the objective compound (104) (6.2 g, 8.3 mmol, 78%).

¹H NMR(CDCl₃, 200 MHz) δ=1.68(s, 6H), 7.28-7.38(m, 12H), 7.54-7.60(m, 7H), 7.67-7.77(m, 12H), 7.84-7.90(m, 3H)

MS/FAB: 749.3(found) 748.3(calculated)

### Preparation Example 5

### Preparation of Compound (105)

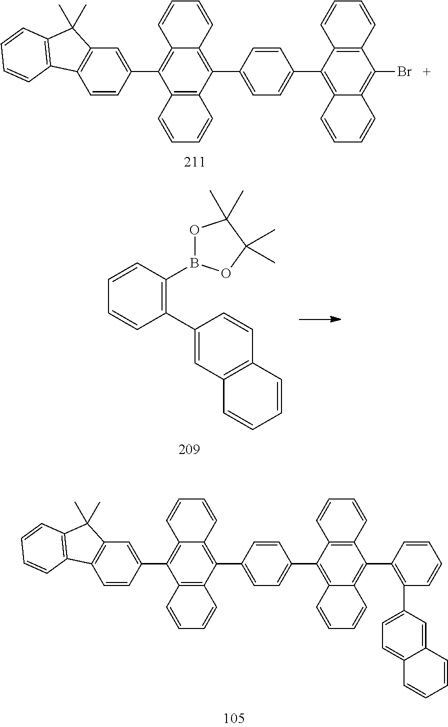

In toluene (100 mL), dissolved were Compound (211) (5.0 g, 7.1 mmol), Compound (209) (2.8 g, 8.5 mmol), tetrakispalladium (O) triphenylphosphine (Pd(PPh₃)₄) (0.8 g, 0.7 mmol) and Aliquat 336 (0.3 mL, 0.7 mmol). To the solution, aqueous 2M sodium carbonate solution (36 mL) was added, and the mixture was stirred at 120° C. under reflux for 4 hours. Then, the mixture was cooled to 25° C., and the reaction was quenched by adding distilled water (100 mL). The reaction mixture was extracted with ethyl acetate (80 mL), and the extract was dried under reduced pressure. Recrystallization from tetrahydrofuran (10 mL) and methanol (100 mL) gave the objective compound (105) (3.2 g, 3.9 mmol, 55%).

¹H NMR(CDCl₃, 200 MHz) δ=1.67(s, 6H), 7.28-7.40(m, 14H), 7.54-7.55(m, 8H), 7.60-7.67(m, 11H), 7.73-7.77(m, 2H), 7.84-7.92(m, 3H)

MS/FAB: 825.3(found) 824.3(calculated)

### Preparation Example 6

### Preparation of Compound (106)

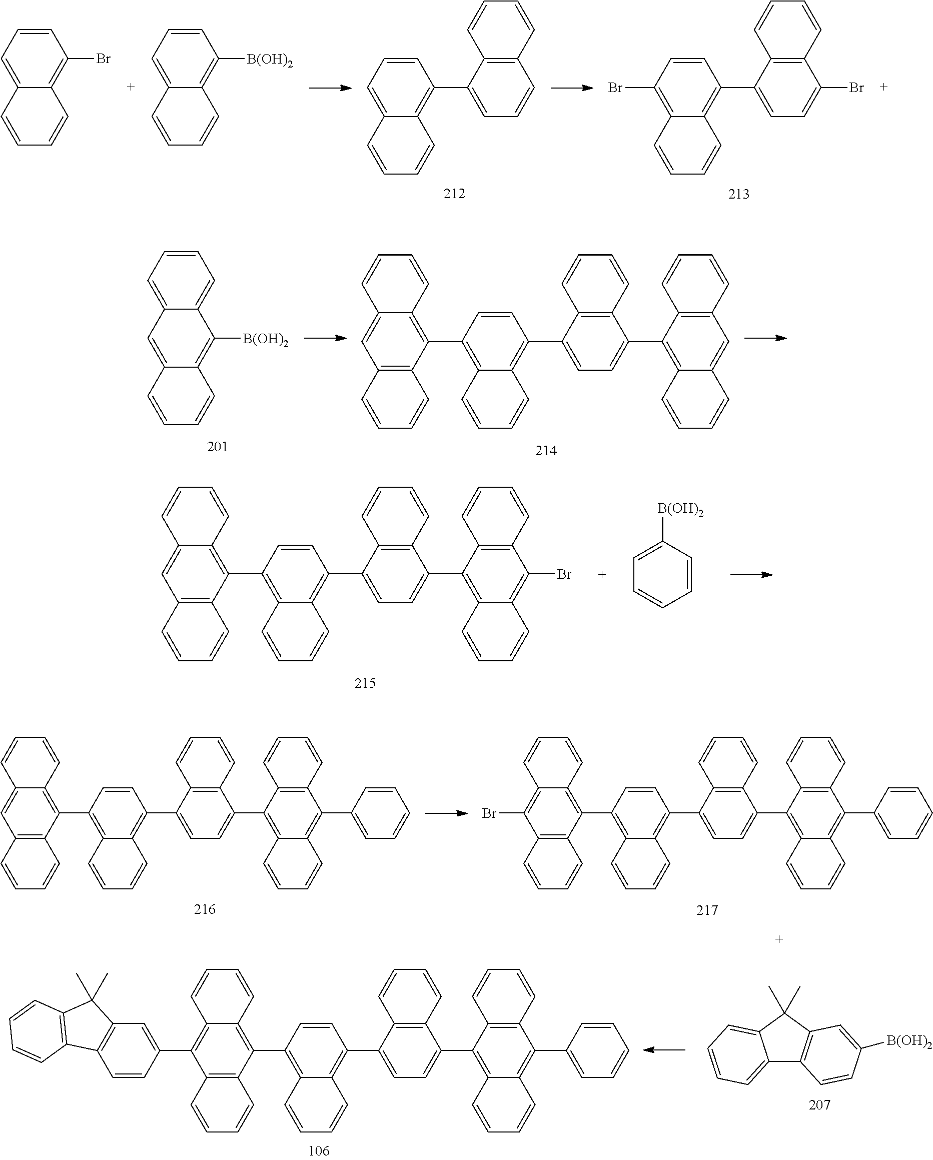

### Preparation of Compound (212)

In toluene (400 mL) and ethanol (200 mL), dissolved were 1-bromonaphthalene (10.0 g, 48.3 mmol), 1-naphthylboronic acid (10.8 g, 62.8 mmol) and tetrakispalladium (O) triphenylphosphine (Pd(PPh₃)₄) (5.5 g, 4.8 mmol). To the solution, the aqueous 2M sodium carbonate solution (240 mL) was added, and the mixture was stirred at 120° C. under reflux for 4 hours. Then, the mixture was cooled to 25° C., and the reaction was quenched by adding distilled water (500 mL). The reaction mixture was extracted with ethyl acetate (300 mL), and the extract was dried under reduced pressure. Recrystallization from tetrahydrofuran (50 mL) and methanol (500 mL) gave the objective compound (212) (9.7 g, 38.1 mmol).

### Preparation of Compound (213)

Compound (212) (9.7 g, 38.1 mmol) was dissolved in dichloromethane (100 mL), and bromine (4.3 mL, 83.8 mmol) solution dissolved in dichloromethane (30 mL) was slowly added dropwise thereto at −5° C. The mixture was stirred at 0° C. for 2 hours, and then at 25° C. for 12 hours. After neutralizing with aqueous potassium hydroxide (KOH) solution, the reaction mixture was extracted with dichloromethane (100 mL), and the extract was dried under reduced pressure and washed with hexane to obtain the objective compound (213) (11.8 g, 28.6 mmol).

### Preparation of Compound (214)

In toluene (300 mL) and ethanol (150 mL), dissolved were Compound (213) (11.8 g, 28.6 mmol), Compound (201) (14.0 g, 62.9 mmol) and tetrakispalladium (O) triphenylphosphine (Pd(PPh₃)₄) (3.4 g, 2.9 mmol). To the solution, aqueous 2M sodium carbonate solution (150 mL) was added, and the mixture was stirred at 120° C. under reflux for one day. Then, the mixture was cooled to 25° C., and the reaction was quenched by adding distilled water (300 mL). The reaction mixture was extracted with ethyl acetate (200 mL), and the extract was dried under reduced pressure. Recrystallization from tetrahydrofuran (30 mL) and methanol (500 mL) gave the objective compound (214) (14.3 g, 23.6 mmol).

### Preparation of Compound (215)

Compound (214) (14.3 g, 23.6 mmol) obtained as above and N-bromosuccinimide (4.6 g, 26.0 mmol) were dissolved in dichloromethane (300 mL) under nitrogen atmosphere, and the solution was stirred at 25° C. for one day. Then, distilled water (400 mL) was added to quench the reaction. The reaction mixture was extracted with dichloromethane (200 mL), and the extract was dried under reduced pressure. Recrystallization from tetrahydrofuran (20 mL) and methanol (400 mL) gave the objective compound (215) (13.5 g, 19.7 mmol).

### Preparation of Compound (216)

In toluene (300 mL) and ethanol (150 mL), dissolved were Compound (215) (13.5 g, 19.7 mmol), phenylboronic acid (2.9 g, 23.6 mmol) and tetrakispalladium (O) triphenylphosphine (Pd(PPh₃)₄) (2.3 g, 2.0 mmol). To the solution, aqueous 2M sodium carbonate solution (100 mL) was added, and the mixture was stirred at 120° C. under reflux for 6 hours. Then, the mixture was cooled to 25° C., and the reaction was quenched by adding distilled water (400 mL). The reaction mixture was extracted with ethyl acetate (300 mL), and the extract was dried under reduced pressure. Recrystallization from tetrahydrofuran (30 mL) and methanol (500 mL) gave the objective compound (216) (10.6 g, 15.5 mmol).

### Preparation of Compound (217)

Compound (216) (10.6 g, 15.5 mmol) and N-bromosuccinimide (3.3 g, 18.6 mmol) were dissolved in dichloromethane (200 mL) under nitrogen atmosphere, and the solution was stirred at 25° C. for one day. Then, distilled water (300 mL) was added to quench the reaction. The reaction mixture was extracted with dichloromethane (150 mL), and the extract was dried under reduced pressure. Recrystallization from tetrahydrofuran (30 mL) and methanol (300 mL) gave the objective compound (217) (8.9 g, 11.7 mmol).

### Preparation of Compound (106)

In toluene (150 mL) and ethanol (70 mL), dissolved were Compound (217) (8.9 g, 11.7 mmol), Compound (207) (3.3 g, 14.0 mmol) and tetrakispalladium (O) triphenylphosphine (Pd(PPh₃)₄) (1.4 g, 1.2 mmol). To the solution, aqueous 2M sodium carbonate solution (60 mL) was added, and the mixture was stirred at 120° C. under reflux for 5 hours. Then, the mixture was cooled to 25° C., and the reaction was quenched by adding distilled water (200 mL). The reaction mixture was extracted with ethyl acetate (100 mL), and the extract was dried under reduced pressure. Recrystallization from tetrahydrofuran (20 mL) and methanol (300 mL) gave the objective compound (106) (6.3 g, 7.2 mmol, 62%).

¹H NMR(CDCl₃, 200 MHz) δ=1.68(s, 6H), 7.22-7.38(m, 17H), 7.55-7.60(m, 6H), 7.67(m, 12H), 7.77(m, 1H), 7.85-7.89(m, 2H)

MS/FAB: 875.3(found) 874.3(calculated)

### Preparation Example 7

### Preparation of Compound (107)

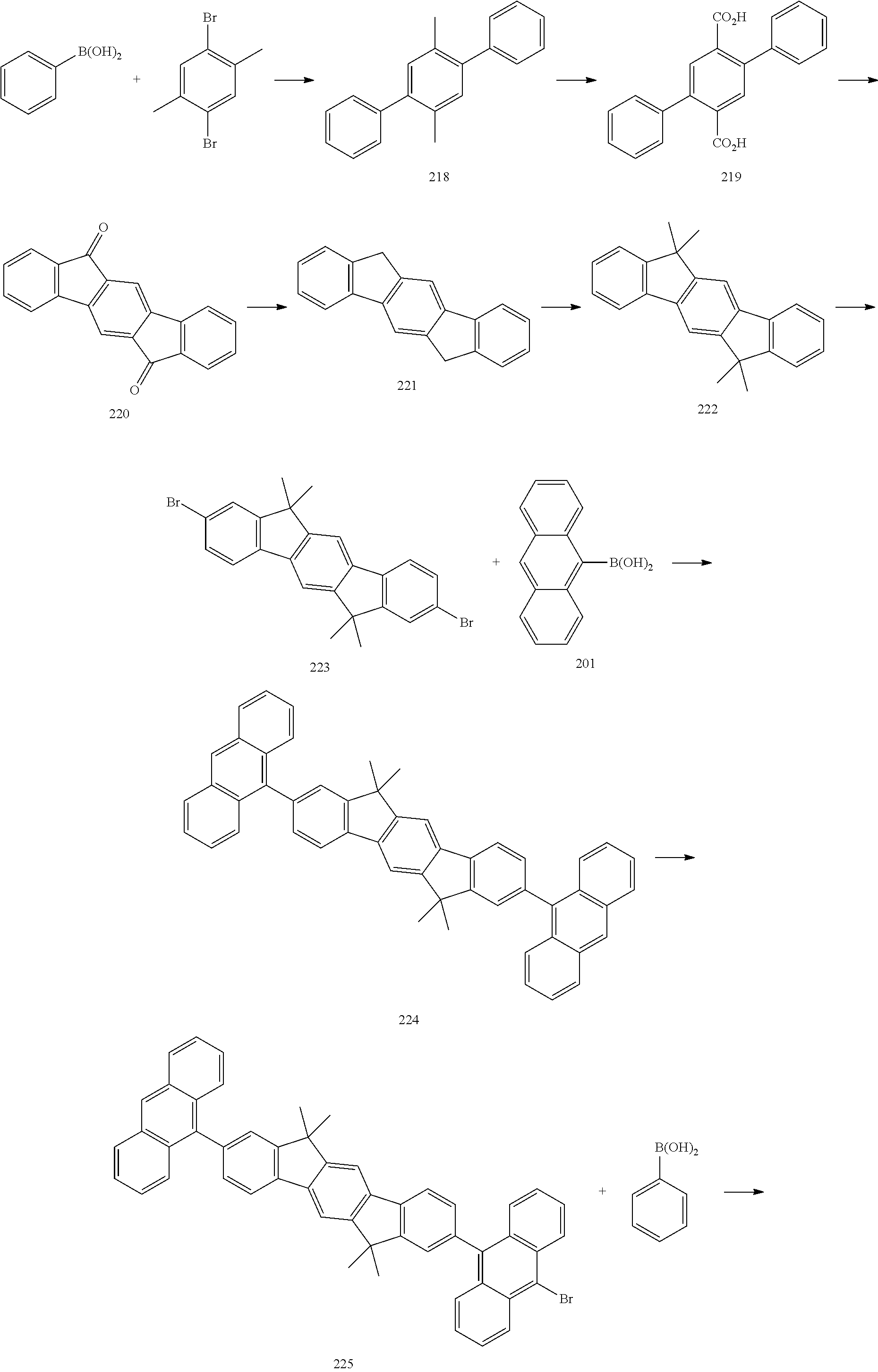

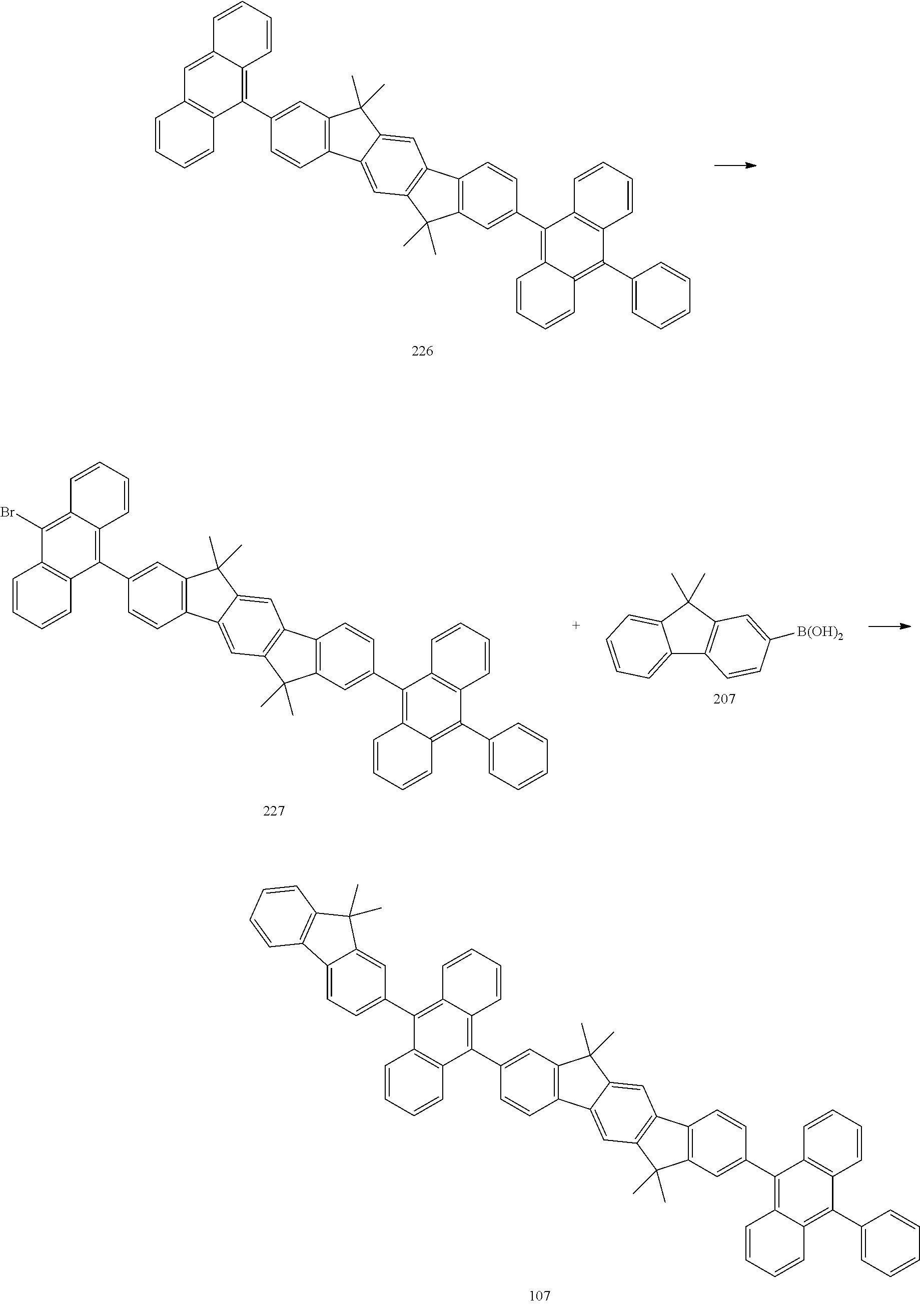

### Preparation of Compound (218)

In water (200 mL), suspended were 1,4-dibromo-p-xylene (20.0 g, 80.0 mmol), phenylboronic acid (20.4 g, 170.0 mmol), Pd(OAc)₂ (0.04 g, 1.5 mmol), potassium carbonate (K₂CO₃) (52.4 g, 380.0 mmol) and n-Bu₄NBr (49.0 g) (150.0 mmol), and the suspension was stirred at 70° C. for 24 hours. When the reaction was completed, distilled water (200 mL) was added to the reaction mixture, and the solid produced was filtered under reduced pressure to obtain Compound (218) (19.6 g, 80 mmol).

### Preparation of Compound (219)

Compound (218) (19.6 g, 80.0 mmol) obtained as above was dissolved in pyridine (120.0 mL, 152.0 mmol) and water (200 mL), and potassium permanganate (42.0 g, 270.0 mmol) was slowly added to the solution by two portions. After heating the mixture under reflux for 48 hours, distilled water (500 mL) at 50° C. was added. The solid byproduct was removed by filtration under reduced pressure. Hydrochloric acid (35%, 300 mL) was added thereto to obtain Compound (219) as solid (17.4 g, 60.0 mmol).

### Preparation of Compound (220)

Compound (219) (17.4 g, 60.0 mmol) obtained as above was added to sulfuric acid (180 mL), and the mixture was stirred at 25° C. for 4 hours. When the reaction was completed, ice water (600 mL) was added thereto, and the resultant mixture was stirred for 1 hour and filtered under reduced pressure. To the solid obtained, added was a solution of potassium carbonate (20 g) in distilled water (500 mL). The mixture was stirred for 1 hour, and filtered under reduced pressure to obtain Compound (220) (12.0 g, 43.0 mmol).

### Preparation of Compound (221)

Compound (220) (12.0 g, 43.0 mmol) obtained as above was dissolved in diethylene glycol (140 mL), and potassium hydroxide (24.0 g, 425.0 mmol) and hydrazine hydrate (20.7 mL, 430 mmol) were added thereto. After stirring at 220° C. for 48 hours, the reaction mixture was cooled to 25° C., and 20% hydrochloric acid solution (400 mL) was added thereto. After stirring for 1 hour, the solid produced was filtered under reduced pressure. Acetone and tetrahydrofuran (200 mL each) were added to the solid, and the mixture was stirred for 20 hours, filtered under reduced pressure and dried under reduced pressure to obtain Compound (221) named as indenofluorene (8.5 g, 33.4 mmol).

### Preparation of Compound (222)

Compound (221) (8.5 g, 33.4 mmol) was dissolved in tetrahydrofuran (100 mL), and the solution was chilled to −78° C. Then, n-butyl lithium (n-BuLi, 1.6M in n-hexane) (48.0 mL, 76.8 mmol) was slowly added dropwise thereto. After 1 hour of reaction, iodomethane (8.2 mL, 86.8 mmol) was added, and the reaction mixture was slowly warmed and stirred at 25° C. for 1 hour. The temperature of the reaction was chilled again to −78° C., and n-BuLi (1.6 M in n-hexane) (60.5 mL, 96.9 mmol) was slowly added dropwise thereto. After 1 hour of reaction, iodomethane (11.1 mL, 116.9 mmol) was added, and the reaction mixture was slowly warmed and stirred at 25° C. for 15 hour. The reaction was quenched by adding aqueous ammonium chloride solution (300 mL) and distilled water (200 mL), and the resultant mixture was extracted with dimethyl chloride (300 mL). The extract was dried over anhydrous magnesium sulfate and filtered. The organic layer thus obtained was evaporated under reduced pressure, and the residue was recrystallized from hexane (500 mL) to obtain Compound (222) (6.3 g, 20.3 mmol).

### Preparation of Compound (223)

Compound (222) (6.3 g, 20.3 mmol) was dissolved in dichloromethane (150 mL), and bromine (2.3 mL, 44.7 mmol) solution dissolved in dichloromethane (30 mL) was slowly added dropwise thereto at −5° C. The mixture was stirred at 0° C. for 2 hours, and then at 25° C. for 12 hours. After neutralizing with aqueous potassium hydroxide (KOH) solution, the reaction mixture was extracted with dichloromethane (200 mL), and the extract was dried under reduced pressure and washed with hexane to obtain the objective compound (223) (5.2 g, 11.1 mmol).

### Preparation of Compound (224)

In toluene (120 mL) and ethanol (60 mL), dissolved were Compound (223) (5.2 g, 11.1 mmol), Compound (201) (5.4 g, 24.4 mmol) and tetrakispalladium (O) triphenylphosphine (Pd(PPh₃)₄) (1.3 g, 1.1 mmol). To the solution, aqueous 2M sodium carbonate solution (60 mL) was added, and the mixture was stirred at 120° C. under reflux for one day. Then, the mixture was cooled to 25° C., and the reaction was quenched by adding distilled water (200 mL). The reaction mixture was extracted with ethyl acetate (100 mL), and the extract was dried under reduced pressure. Recrystallization from tetrahydrofuran (20 mL) and methanol (200 mL) gave the objective compound (224) (6.5 g, 9.8 mmol).

### Preparation of Compound (225)

Compound (224) (6.5 g, 9.8 mmol) obtained as above and N-bromosuccinimide (1.9 g, 10.8 mmol) were dissolved in dichloromethane (200 mL) under nitrogen atmosphere, and the solution was stirred at 25° C. for one day. Then, distilled water (200 mL) was added to quench the reaction. The reaction mixture was extracted with dichloromethane (100 mL), and the extract was dried under reduced pressure. Recrystallization from tetrahydrofuran (20 mL) and methanol (200 mL) gave the objective compound (225) (5.6 g, 7.5 mmol).

### Preparation of Compound (226)

In toluene (100 mL) and ethanol (50 mL), dissolved were Compound (225) (5.6 g, 7.5 mmol), phenylboronic acid (1.1 g, 9.0 mmol) and tetrakispalladium (O) triphenylphosphine (Pd(PPh₃)₄) (0.9 g, 0.8 mmol). To the solution, aqueous 2M sodium carbonate solution (40 mL) was added, and the mixture was stirred at 120° C. under reflux for 4 hours. Then, the mixture was cooled to 25° C., and the reaction was quenched by adding distilled water (200 mL). The reaction mixture was extracted with ethyl acetate (100 mL), and the extract was dried under reduced pressure. Recrystallization from tetrahydrofuran (20 mL) and methanol (150 mL) gave the objective compound (226) (4.2 g, 5.7 mmol).

### Preparation of Compound (227)

Compound (226) (4.2 g, 5.7 mmol) obtained as above and N-bromosuccinimide (1.2 g, 6.8 mmol) were dissolved in dichloromethane (100 mL) under nitrogen atmosphere, and the solution was stirred at 25° C. for one day. Then, distilled water (150 mL) was added to quench the reaction. The reaction mixture was extracted with dichloromethane (100 mL), and the extract was dried under reduced pressure. Recrystallization from tetrahydrofuran (10 mL) and methanol (100 mL) gave the objective compound (227) (3.1 g, 3.8 mmol).

### Preparation of Compound (107)

In toluene (100 mL) and ethanol (50 mL), dissolved were Compound (227) (3.1 g, 3.8 mmol), Compound (207) (1.1 g, 4.6 mmol) and tetrakispalladium (O) triphenylphosphine (Pd(PPh₃)₄) (0.5 g, 0.4 mmol). To the solution, aqueous 2M sodium carbonate solution (20 mL) was added, and the mixture was stirred at 120° C. under reflux for 5 hours. Then, the mixture was cooled to 25° C., and the reaction was quenched by adding distilled water (100 mL). The reaction mixture was extracted with ethyl acetate (50 mL), and the extract was dried under reduced pressure. Recrystallization from tetrahydrofuran (20 mL) and methanol (200 mL) gave the objective compound (107) (1.7 g, 1.8 mmol, 49%).

¹H NMR(CDCl₃, 200 MHz) δ=1.68 (s, 18H), 7.23-7.39(m, 13H), 7.48(m, 2H), 7.55-7.69(m, 11H), 7.77(m, 3H), 7.83-7.90(m, 4H), 8.12(m, 1H)

MS/FAB: 931.4(found) 930.4(calculated)

### Preparation Example 8

### Preparation of Compound (108)

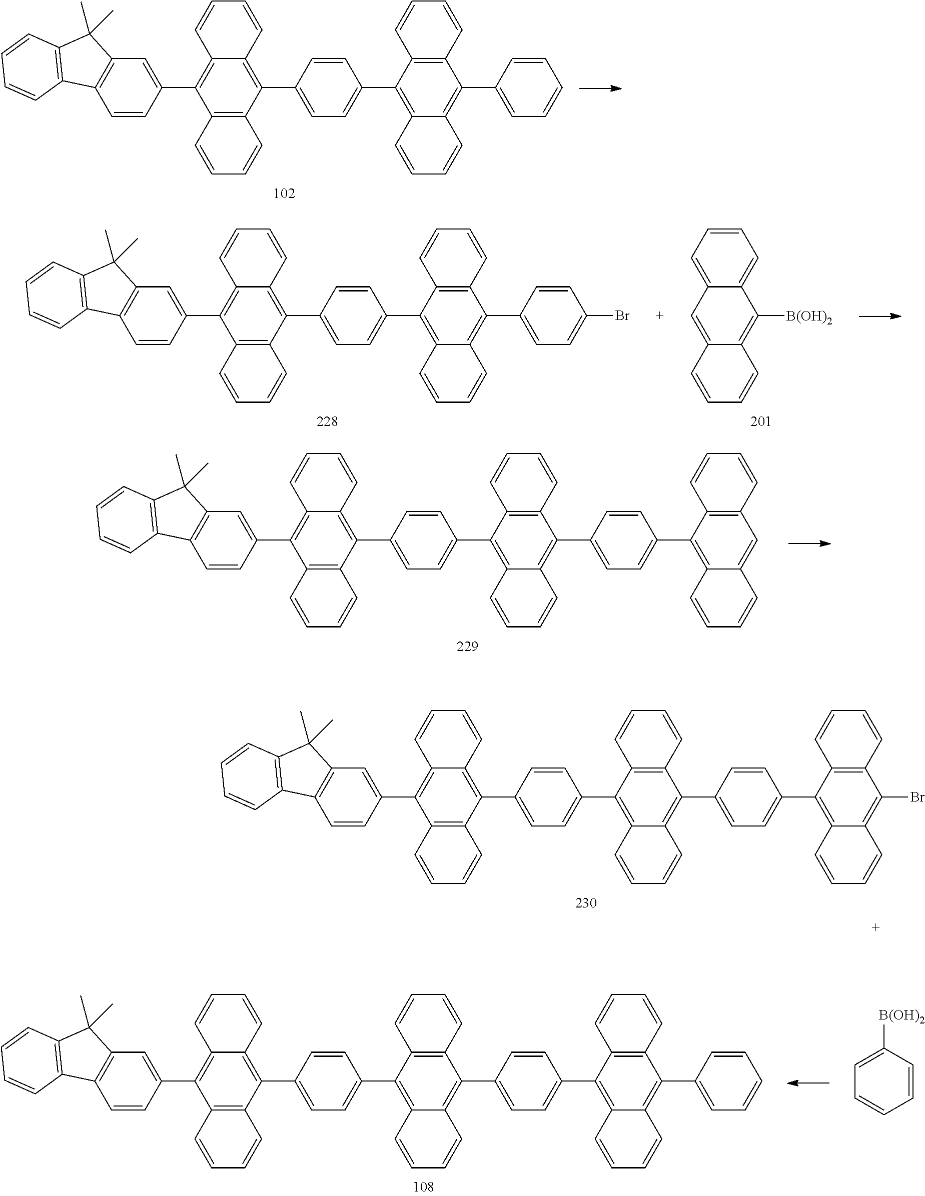

### Preparation of Compound (228)

Compound (102) (5.0 g, 7.2 mmol) and N-bromosuccinimide (1.4 g, 7.9 mmol) were dissolved in dichloromethane (100 mL) under nitrogen atmosphere, and the solution was stirred at 25° C. for one day. Then, distilled water (200 mL) was added to quench the reaction. The reaction mixture was extracted with dichloromethane (100 mL), and the extract was dried under reduced pressure. Recrystallization from tetrahydrofuran (10 mL) and methanol (200 mL) gave the objective compound (228) (4.1 g, 5.3 mmol).

### Preparation of Compound (229)

In toluene (100 mL) and ethanol (50 mL), dissolved were Compound (228) (4.1 g, 5.3 mmol), Compound (201) (1.5 g, 6.9 mmol) and tetrakispalladium (O) triphenylphosphine (Pd(PPh₃)₄) (0.6 g, 0.5 mmol). To the solution, aqueous 2M sodium carbonate solution (30 mL) was added, and the mixture was stirred at 120° C. under reflux for 4 hours. Then, the mixture was cooled to 25° C., and the reaction was quenched by adding distilled water (150 mL). The reaction mixture was extracted with ethyl acetate (60 mL), and the extract was dried under reduced pressure. Recrystallization from tetrahydrofuran (10 mL) and methanol (200 mL) gave the objective compound (229) (2.8 g, 3.2 mmol).

### Preparation of Compound (230)

Compound (229) (2.8 g, 3.2 mmol) and N-bromosuccinimide (0.7 g, 3.8 mmol) were dissolved in dichloromethane (50 mL) under nitrogen atmosphere, and the solution was stirred at 25° C. for one day. Then, distilled water (100 mL) was added to quench the reaction. The reaction mixture was extracted with dichloromethane (50 mL), and the extract was dried under reduced pressure. Recrystallization from tetrahydrofuran (5 mL) and methanol (100 mL) gave the objective compound (230) (2.3 g, 2.4 mmol).

### Preparation of Compound (108)

In toluene (50 mL) and ethanol (25 mL), dissolved were Compound (230) (2.3 g, 2.4 mmol), phenylboronic acid (0.4 g, 3.1 mmol) and tetrakispalladium (O) triphenylphosphine (Pd(PPh₃)₄) (0.3 g, 0.2 mmol). To the solution, aqueous 2M sodium carbonate solution (12 mL) was added, and the mixture was stirred at 120° C. under reflux for 4 hours. Then, the mixture was cooled to 25° C., and the reaction was quenched by adding distilled water (60 mL). The reaction mixture was extracted with ethyl acetate (50 mL), and the extract was dried under reduced pressure. Recrystallization from tetrahydrofuran (5 mL) and methanol (80 mL) gave the objective compound (108) (1.5 g, 1.6 mmol, 68%).

¹H NMR(CDCl₃, 200 MHz) δ=1.67(s, 6H), 7.23-7.38(m, 17H), 7.48-7.67(m, 24H), 7.77(m, 1H), 7.84-7.92(m, 2H)

MS/FAB: 951.3(found) 950.3(calculated)

### Preparation Example 9

### Preparation of Compound (109)

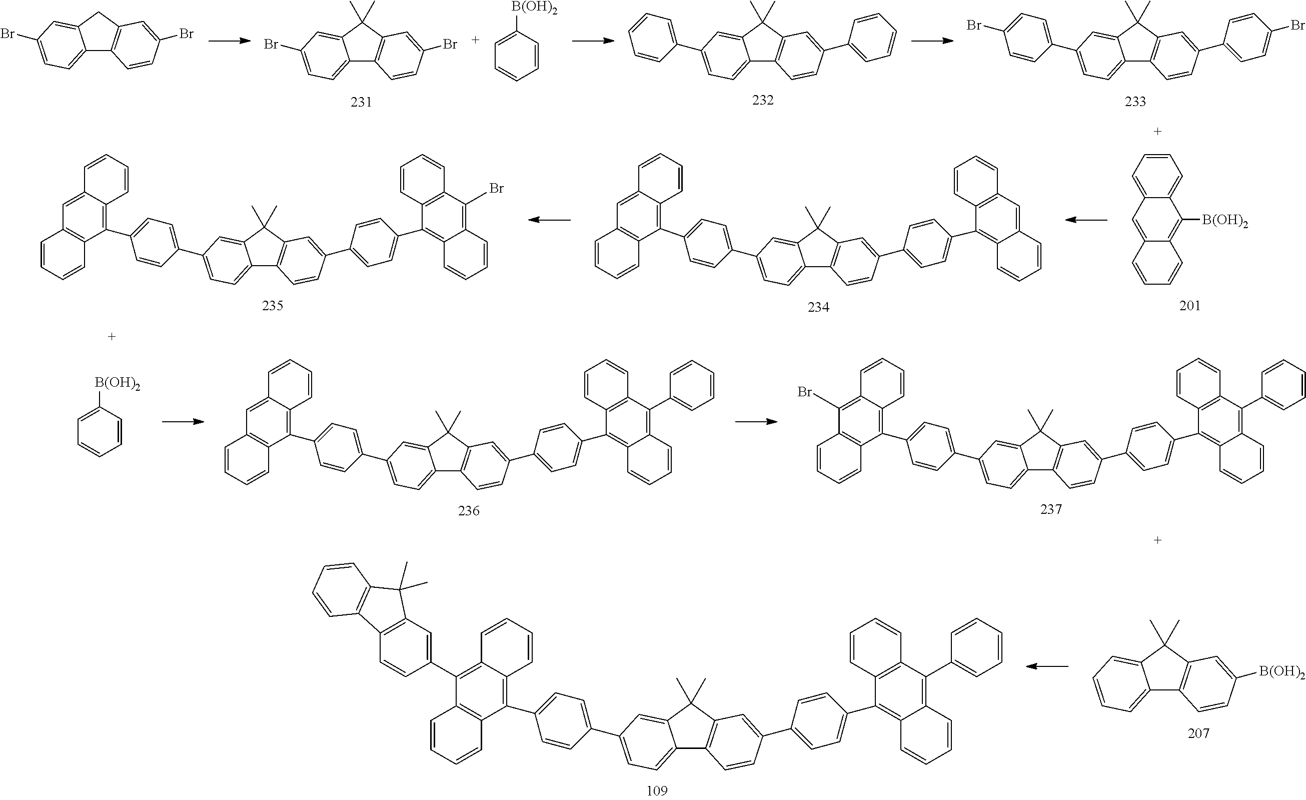

### Preparation of Compound (231)

In dimethylsulfoxide (200 mL), dissolved were 2,7-dibromofluorene (10.0 g, 30.9 mmol) and potassium hydroxide (KOH) (10.4 g, 185.4 mmol). Iodomethane (7.7 mL, 123.6 mmol) was added thereto at 10° C., and the temperature was raised to 30° C. After stirring for 12 hours, the reaction mixture was poured into distilled water, and the mixture was stirred for 30 minutes. The solid produced was filtered under reduced pressure and washed with hexane to obtain the objective compound (231) (8.6 g, 24.4 mmol).

### Preparation of Compound (232)

In toluene (300 mL) and ethanol (150 mL), dissolved were Compound (231) (8.6 g, 24.4 mmol), phenylboronic acid (6.5 g, 53.7 mmol) and tetrakispalladium (O) triphenylphosphine (Pd(PPh₃)₄) (2.8 g, 2.4 mmol). To the solution, aqueous 2M sodium carbonate solution (120 mL) was added, and the mixture was stirred at 120° C. under reflux for one day. Then, the mixture was cooled to 25° C., and the reaction was quenched by adding distilled water (300 mL). The reaction mixture was extracted with ethyl acetate (300 mL), and the extract was dried under reduced pressure. Recrystallization from tetrahydrofuran (20 mL) and methanol (300 mL) gave the objective compound (232) (7.2 g, 20.8 mmol).

### Preparation of Compound (233)

Compound (232) (7.2 g, 20.8 mmol) was dissolved in dichloromethane (300 mL), and bromine (2.4 mL, 45.8 mmol) solution dissolved in dichloromethane (30 mL) was slowly added dropwise thereto at −5° C. The mixture was stirred at 0° C. for 2 hours, and then at 25° C. for 12 hours. After neutralizing with aqueous potassium hydroxide (KOH) solution, the reaction mixture was extracted with dichloromethane (100 mL), and the extract was dried under reduced pressure and washed with hexane to obtain the objective compound (233) (8.6 g, 17.1 mmol).

### Preparation of Compound (234)

In toluene (200 mL) and ethanol (100 mL), dissolved were Compound (233) (8.6 g, 17.1 mmol), Compound (201) (8.4 g, 37.6 mmol) and tetrakispalladium (O) triphenylphosphine (Pd(PPh₃)₄) (2.0 g, 1.7 mmol). To the solution, aqueous 2M sodium carbonate solution (100 mL) was added, and the mixture was stirred at 120° C. under reflux for one day. Then, the mixture was cooled to 25° C., and the reaction was quenched by adding distilled water (300 mL). The reaction mixture was extracted with ethyl acetate (100 mL), and the extract was dried under reduced pressure. Recrystallization from tetrahydrofuran (30 mL) and methanol (300 mL) gave the objective compound (234) (9.4 g, 13.4 mmol).

### Preparation of Compound (235)

Compound (234) (9.4 g, 13.4 mmol) and N-bromosuccinimide (2.6 g, 14.7 mmol) were dissolved in dichloromethane (200 mL) under nitrogen atmosphere, and the solution was stirred at 25° C. for one day. Then, distilled water (300 mL) was added to quench the reaction. The reaction mixture was extracted with dichloromethane (100 mL), and the extract was dried under reduced pressure. Recrystallization from tetrahydrofuran (30 mL) and methanol (400 mL) gave the objective compound (235) (7.8 g, 10.0 mmol).

### Preparation of Compound (236)

In toluene (100 mL) and ethanol (50 mL), dissolved were Compound (235) (7.8 g, 10.0 mmol), phenylboronic acid (1.5 g, 12.0 mmol) and tetrakispalladium (O) triphenylphosphine (Pd(PPh₃)₄) (1.2 g, 1.0 mmol). To the solution, aqueous 2M sodium carbonate solution (50 mL) was added, and the mixture was stirred at 120° C. under reflux for 4 hours. Then, the mixture was cooled to 25° C., and the reaction was quenched by adding distilled water (150 mL). The reaction mixture was extracted with ethyl acetate (100 mL), and the extract was dried under reduced pressure. Recrystallization from tetrahydrofuran (15 mL) and methanol (150 mL) gave the objective compound (236) (4.9 g, 6.3 mmol).

### Preparation of Compound (237)

Compound (236) (4.9 g, 6.3 mmol) obtained as above and N-bromosuccinimide (1.3 g, 7.6 mmol) were dissolved in dichloromethane (100 mL) under nitrogen atmosphere, and the solution was stirred at 25° C. for one day. Then, distilled water (150 mL) was added to quench the reaction. The reaction mixture was extracted with dichloromethane (100 mL), and the extract was dried under reduced pressure. Recrystallization from tetrahydrofuran (10 mL) and methanol (150 mL) gave the objective compound (237) (3.6 g, 4.2 mmol).

### Preparation of Compound (109)

In toluene (100 mL) and ethanol (50 mL), dissolved were Compound (237) (3.6 g, 4.2 mmol), Compound (207) (1.2 g, 5.0 mmol) and tetrakispalladium (O) triphenylphosphine (Pd(PPh₃)₄) (0.5 g, 0.4 mmol). To the solution, aqueous 2M sodium carbonate solution (20 mL) was added, and the mixture was stirred at 120° C. under reflux for 5 hours. Then, the mixture was cooled to 25° C., and the reaction was quenched by adding distilled water (100 mL). The reaction mixture was extracted with ethyl acetate (50 mL), and the extract was dried under reduced pressure. Recrystallization from tetrahydrofuran (20 mL) and methanol (200 mL) gave the objective compound (109) (2.5 g, 2.6 mmol, 63%).

¹H NMR(CDCl₃, 200 MHz) δ=1.67 (s, 12H), 7.20-7.34(m, 13H), 7.48(m, 2H), 7.54-7.67(m, 20H), 7.77-7.78(m, 3H), 7.84-7.90(m, 4H)

MS/FAB: 967.4(found) 966.4(calculated)

### Preparation Example 10

### Preparation of Compound (110)

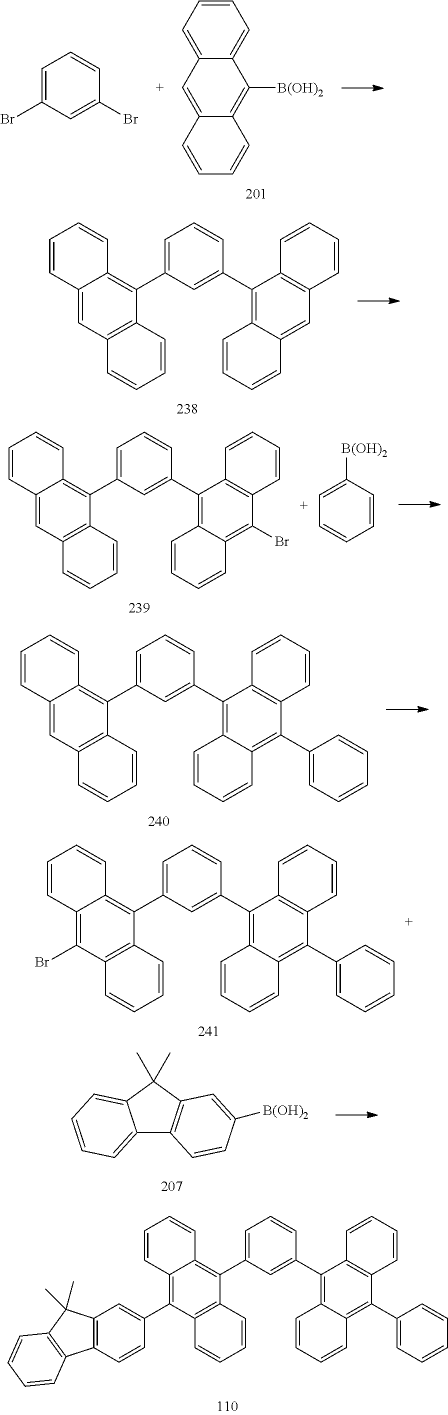

### Preparation of Compound (238)

In toluene (400 mL) and ethanol (200 mL), dissolved were 1,3-dibromobenzene (10.0 g, 42.4 mmol), Compound (201) (20.7 g, 93.3 mmol) and tetrakispalladium (O) triphenylphosphine (Pd(PPh₃)₄) (4.9 g, 4.2 mmol). To the solution, aqueous 2M sodium carbonate solution (212 mL) was added, and the mixture was stirred at 120° C. under reflux for one day. Then, the mixture was cooled to 25° C., and the reaction was quenched by adding distilled water (500 mL). The reaction mixture was extracted with ethyl acetate (300 mL), and the extract was dried under reduced pressure. Recrystallization from tetrahydrofuran (50 mL) and methanol (500 mL) gave the objective compound (238) (12.6 g, 29.3 mmol).

### Preparation of Compound (239)

Compound (238) (12.6 g, 29.3 mmol) and N-bromosuccinimide (5.7 g, 32.2 mmol) were dissolved in dichloromethane (300 mL) under nitrogen atmosphere, and the solution was stirred at 25° C. for one day. Then, distilled water (400 mL) was added to quench the reaction. The reaction mixture was extracted with dichloromethane (200 mL), and the extract was dried under reduced pressure. Recrystallization from tetrahydrofuran (50 mL) and methanol (500 mL) gave the objective compound (239) (11.5 g, 22.6 mmol).

### Preparation of Compound (240)

In toluene (200 mL) and ethanol (100 mL), dissolved were Compound (239) (11.5 g, 22.6 mmol), phenylboronic acid (3.3 g, 27.1 mmol) and tetrakispalladium (O) triphenylphosphine (Pd(PPh₃)₄) (2.7 g, 2.3 mmol). To the solution, aqueous 2M sodium carbonate solution (113 mL) was added, and the mixture was stirred at 120° C. under reflux for 4 hours. Then, the mixture was cooled to 25° C., and the reaction was quenched by adding distilled water (300 mL). The reaction mixture was extracted with ethyl acetate (200 mL), and the extract was dried under reduced pressure. Recrystallization from tetrahydrofuran (40 mL) and methanol (300 mL) gave the objective compound (240) (8.4 g, 16.6 mmol).

### Preparation of Compound (241)

Compound (240) (8.4 g, 16.6 mmol) and N-bromosuccinimide (3.5 g, 19.9 mmol) were dissolved in dichloromethane (200 mL) under nitrogen atmosphere, and the solution was stirred at 25° C. for one day. Then, distilled water (300 mL) was added to quench the reaction. The reaction mixture was extracted with dichloromethane (200 mL), and the extract was dried under reduced pressure. Recrystallization from tetrahydrofuran (20 mL) and methanol (300 mL) gave the objective compound (241) (5.9 g, 10.1 mmol).

### Preparation of Compound (110)

In toluene (100 mL) and ethanol (50 mL), dissolved were Compound (241) (5.9 g, 10.1 mmol), Compound (207) (2.9 g, 12.1 mmol) and tetrakispalladium (O) triphenylphosphine (Pd(PPh₃)₄) (1.2 g, 1.0 mmol). To the solution, aqueous 2M sodium carbonate solution (50 mL) was added, and the mixture was stirred at 120° C. under reflux for 5 hours. Then, the mixture was cooled to 25° C., and the reaction was quenched by adding distilled water (100 mL). The reaction mixture was extracted with ethyl acetate (50 mL), and the extract was dried under reduced pressure. Recrystallization from tetrahydrofuran (20 mL) and methanol (200 mL) gave the objective compound (110) (4.6 g, 6.6 mmol, 66%).

¹H NMR(CDCl₃, 200 MHz) δ=1.68(s, 6H), 7.23-7.32(m, 12H), 7.38-7.48(m, 6H), 7.55-7.70(m, 11H), 7.77(m, 1H), 7.85-7.91(m, 2H)

MS/FAB: 699.3(found) 698.3(calculated)

### Preparation Example 11

### Preparation of Compound (111)

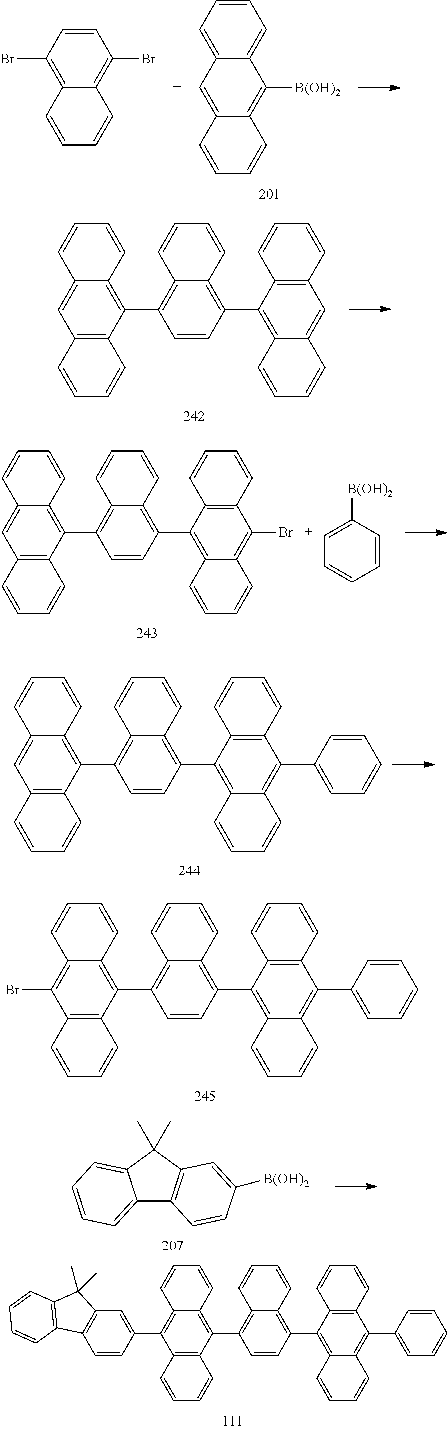

### Preparation of Compound (242)

In toluene (400 mL) and ethanol (200 mL), dissolved were 1,4-dibromonaphthalene (10.0 g, 34.9 mmol), Compound (201) (17.1 g, 76.9 mmol) and tetrakispalladium (O) triphenylphosphine (Pd(PPh₃)₄) (4.0 g, 3.5 mmol). To the solution, aqueous 2M sodium carbonate solution (175 mL) was added, and the mixture was stirred at 120° C. under reflux for one day. Then, the mixture was cooled to 25° C., and the reaction was quenched by adding distilled water (500 mL). The reaction mixture was extracted with ethyl acetate (200 mL), and the extract was dried under reduced pressure. Recrystallization from tetrahydrofuran (50 mL) and methanol (500 mL) gave the objective compound (242) (13.8 g, 28.7 mmol).

### Preparation of Compound (243)

Compound (242) (13.8 g, 28.7 mmol) and N-bromosuccinimide (5.6 g, 31.6 mmol) were dissolved in dichloromethane (300 mL) under nitrogen atmosphere, and the solution was stirred at 25° C. for one day. Then, distilled water (400 mL) was added to quench the reaction. The reaction mixture was extracted with dichloromethane (200 mL), and the extract was dried under reduced pressure. Recrystallization from tetrahydrofuran (40 mL) and methanol (500 mL) gave the objective compound (243) (12.6 g, 22.5 mmol).

### Preparation of Compound (244)

In toluene (300 mL) and ethanol (150 mL), dissolved were Compound (243) (12.6 g, 22.5 mmol), phenylboronic acid (3.3 g, 27.0 mmol) and tetrakispalladium (O) triphenylphosphine (Pd(PPh₃)₄) (2.7 g, 2.3 mmol). To the solution, aqueous 2M sodium carbonate solution (113 mL) was added, and the mixture was stirred at 120° C. under reflux for 4 hours. Then, the mixture was cooled to 25° C., and the reaction was quenched by adding distilled water (400 mL). The reaction mixture was extracted with ethyl acetate (200 mL), and the extract was dried under reduced pressure. Recrystallization from tetrahydrofuran (30 mL) and methanol (500 mL) gave the objective compound (244) (9.4 g, 16.9 mmol).

### Preparation of Compound (245)

Compound (244) (9.4 g, 16.9 mmol) and N-bromosuccinimide (3.6 g, 20.3 mmol) were dissolved in dichloromethane (200 mL) under nitrogen atmosphere, and the solution was stirred at 25° C. for one day. Then, distilled water (300 mL) was added to quench the reaction. The reaction mixture was extracted with dichloromethane (100 mL), and the extract was dried under reduced pressure. Recrystallization from tetrahydrofuran (20 mL) and methanol (300 mL) gave the objective compound (245) (8.2 g, 12.9 mmol).

### Preparation of Compound (111)

In toluene (150 mL) and ethanol (75 mL), dissolved were Compound (245) (8.2 g, 12.9 mmol), Compound (207) (3.7 g, 15.5 mmol) and tetrakispalladium (O) triphenylphosphine (Pd(PPh₃)₄) (1.5 g, 1.3 mmol). To the solution, aqueous 2M sodium carbonate solution (70 mL) was added, and the mixture was stirred at 120° C. under reflux for 5 hours. Then, the mixture was cooled to 25° C., and the reaction was quenched by adding distilled water (200 mL). The reaction mixture was extracted with ethyl acetate (100 mL), and the extract was dried under reduced pressure. Recrystallization from tetrahydrofuran (20 mL) and methanol (300 mL) gave the objective compound (111) (5.3 g, 7.1 mmol, 55%).

¹H NMR(CDCl₃, 200 MHz) δ=1.67(s, 6H), 7.28-7.38(m, 15H), 7.49-7.50(m, 2H), 7.60-7.68(m, 14H), 7.77(m, 1H), 7.85-7.89(m, 2H)

MS/FAB: 749.3(found) 748.3(calculated)

### Preparation Example 12

### Preparation of Compound (112)

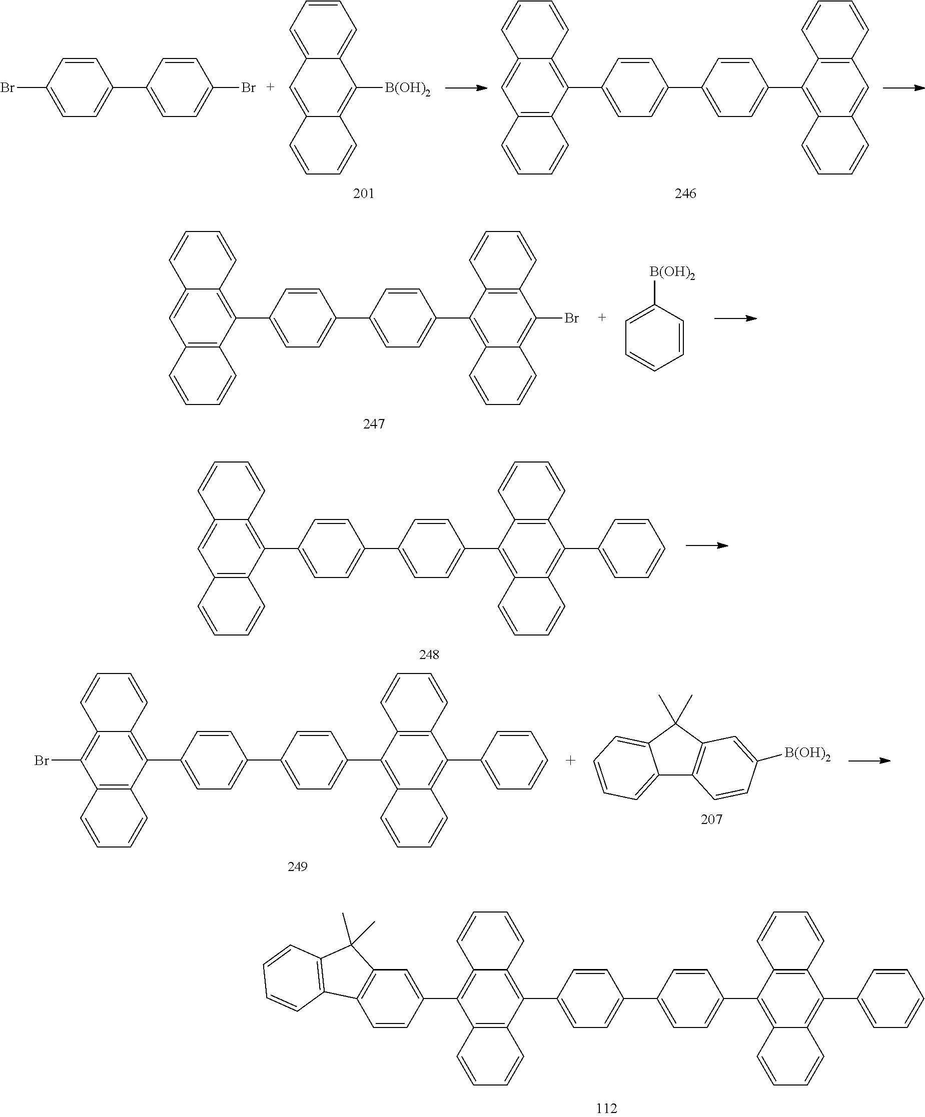

### Preparation of Compound (246)

In toluene (400 mL) and ethanol (200 mL), dissolved were 1,4-biphenyl (10.0 g, 32.1 mmol), Compound (201) (15.7 g, 70.5 mmol) and tetrakispalladium (O) triphenylphosphine (Pd(PPh₃)₄) (3.7 g, 3.2 mmol). To the solution, aqueous 2M sodium carbonate solution (160 mL) was added, and the mixture was stirred at 120° C. under reflux for one day. Then, the mixture was cooled to 25° C., and the reaction was quenched by adding distilled water (500 mL). The reaction mixture was extracted with ethyl acetate (200 mL), and the extract was dried under reduced pressure. Recrystallization from tetrahydrofuran (50 mL) and methanol (500 mL) gave the objective compound (246) (14.3 g, 28.2 mmol).

### Preparation of Compound (247)

Compound (246) (14.3 g, 28.2 mmol) and N-bromosuccinimide (5.5 g, 31.0 mmol) were dissolved in dichloromethane (300 mL) under nitrogen atmosphere, and the solution was stirred at 25° C. for one day. Then, distilled water (400 mL) was added to quench the reaction. The reaction mixture was extracted with dichloromethane (200 mL), and the extract was dried under reduced pressure. Recrystallization from tetrahydrofuran (40 mL) and methanol (500 mL) gave the objective compound (247) (12.8 g, 21.9 mmol).

### Preparation of Compound (248)

In toluene (200 mL) and ethanol (100 mL), dissolved were Compound (247) (12.8 g, 21.9 mmol), phenylboronic acid (3.2 g, 26.2 mmol) and tetrakispalladium (O) triphenylphosphine (Pd(PPh₃)₄) (2.5 g, 2.2 mmol). To the solution, aqueous 2M sodium carbonate solution (110 mL) was added, and the mixture was stirred at 120° C. under reflux for 4 hours. Then, the mixture was cooled to 25° C., and the reaction was quenched by adding distilled water (300 mL). The reaction mixture was extracted with ethyl acetate (200 mL), and the extract was dried under reduced pressure. Recrystallization from tetrahydrofuran (30 mL) and methanol (500 mL) gave the objective compound (248) (10.2 g, 17.5 mmol).

### Preparation of Compound (249)

Compound (248) (10.2 g, 17.5 mmol) and N-bromosuccinimide (3.7 g, 21.0 mmol) were dissolved in dichloromethane (200 mL) under nitrogen atmosphere, and the solution was stirred at 25° C. for one day. Then, distilled water (300 mL) was added to quench the reaction. The reaction mixture was extracted with dichloromethane (100 mL), and the extract was dried under reduced pressure. Recrystallization from tetrahydrofuran (20 mL) and methanol (300 mL) gave the objective compound (249) (8.6g, 13.0 mmol).

### Preparation of Compound (112)

In toluene (150 mL) and ethanol (75 mL), dissolved were Compound (249) (8.6 g, 13.0 mmol), Compound (207) (3.7 g, 15.6 mmol) and tetrakispalladium (O) triphenylphosphine (Pd(PPh₃)₄) (1.5 g, 1.3 mmol). To the solution, aqueous 2M sodium carbonate solution (65 mL) was added, and the mixture was stirred at 120° C. under reflux for 5 hours. Then, the mixture was cooled to 25° C., and the reaction was quenched by adding distilled water (200 mL). The reaction mixture was extracted with ethyl acetate (100 mL), and the extract was dried under reduced pressure. Recrystallization from tetrahydrofuran (20 mL) and methanol (300 mL) gave the objective compound (112) (7.2 g, 9.3 mmol, 71%).

¹H NMR(CDCl₃, 200 MHz) δ=1.68(s, 6H), 7.24-7.39(m, 13H), 7.48-7.60(m, 12H), 7.67(m, 8H), 7.77-7.90(m, 3H)

MS/FAB: 775.3(found) 774.3(calculated)

### Preparation Example 13

### Preparation of Compound (113)

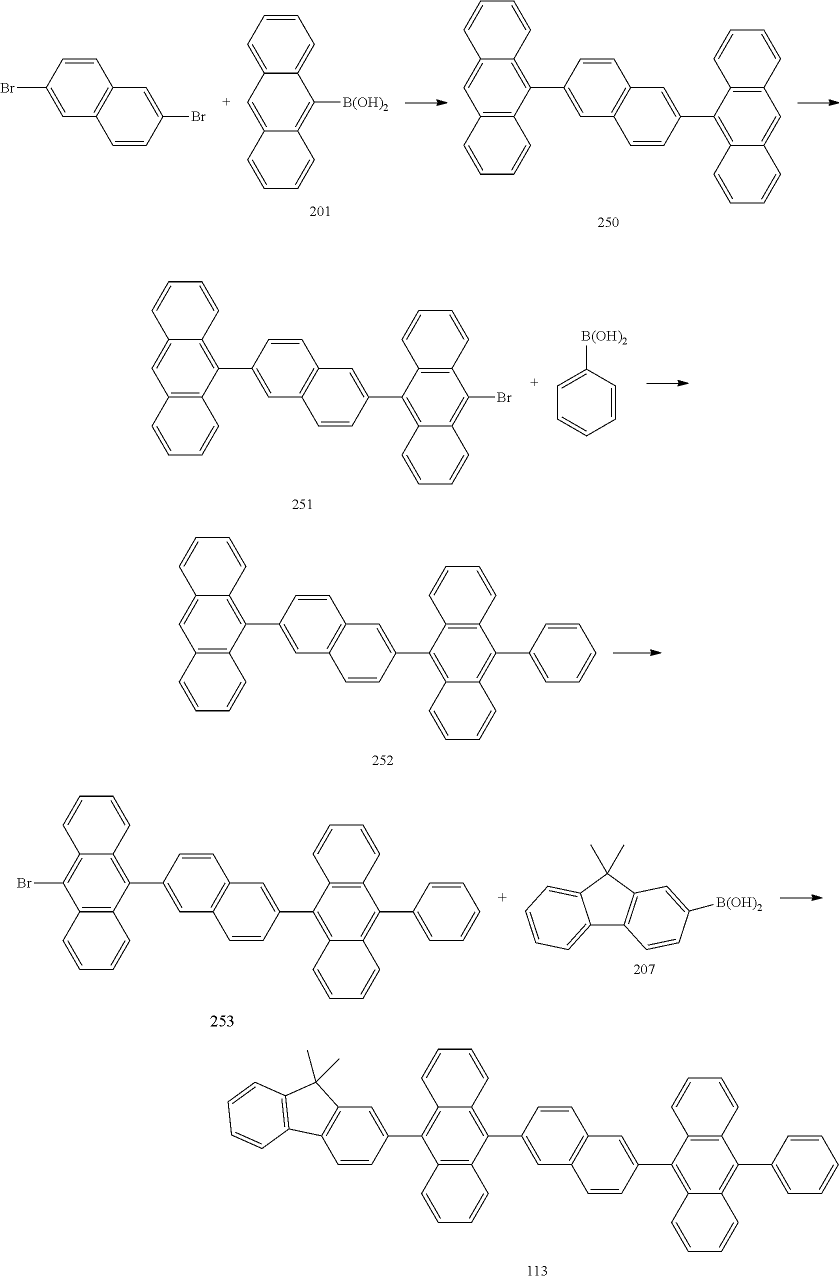

### Preparation of Compound (250)

In toluene (400 mL) and ethanol (200 mL), dissolved were 2,6-dibromonaphthalene (10.0 g, 35.0 mmol), Compound (201) (17.1 g, 76.9 mmol) and tetrakispalladium (O) triphenylphosphine (Pd(PPh₃)₄) (4.0 g, 3.5 mmol). To the solution, aqueous 2M sodium carbonate solution (175 mL) was added, and the mixture was stirred at 120° C. under reflux for one day. Then, the mixture was cooled to 25° C., and the reaction was quenched by adding distilled water (500 mL). The reaction mixture was extracted with ethyl acetate (200 mL), and the extract was dried under reduced pressure. Recrystallization from tetrahydrofuran (50 mL) and methanol (500 mL) gave the objective compound (250) (13.7 g, 28.5 mmol).

### Preparation of Compound (251)

Compound (250) (13.7 g, 28.5 mmol) and N-bromosuccinimide (9.1 g, 51.3 mmol) were dissolved in dichloromethane (300 mL) under nitrogen atmosphere, and the solution was stirred at 25° C. for one day. Then, distilled water (400 mL) was added to quench the reaction. The reaction mixture was extracted with dichloromethane (200 mL), and the extract was dried under reduced pressure. Recrystallization from tetrahydrofuran (40 mL) and methanol (500 mL) gave the objective compound (251) (12.1 g, 21.6 mmol).

### Preparation of Compound (252)

In toluene (200 mL) and ethanol (100 mL), dissolved were Compound (251) (12.1 g, 21.6 mmol), phenylboronic acid (3.2 g, 26.0 mmol) and tetrakispalladium (O) triphenylphosphine (Pd(PPh₃)₄) (2.5 g, 2.2 mmol). To the solution, aqueous 2M sodium carbonate solution (108 mL) was added, and the mixture was stirred at 120° C. under reflux for 4 hours. Then, the mixture was cooled to 25° C., and the reaction was quenched by adding distilled water (300 mL). The reaction mixture was extracted with ethyl acetate (200 mL), and the extract was dried under reduced pressure. Recrystallization from tetrahydrofuran (30 mL) and methanol (500 mL) gave the objective compound (252) (8.4 g, 15.1 mmol).

### Preparation of Compound (253)

Compound (252) (8.4 g, 15.1 mmol) and N-bromosuccinimide (4.8 g, 27.2 mmol) were dissolved in dichloromethane (150 mL) under nitrogen atmosphere, and the solution was stirred at 25° C. for one day. Then, distilled water (200 mL) was added to quench the reaction. The reaction mixture was extracted with dichloromethane (100 mL), and the extract was dried under reduced pressure. Recrystallization from tetrahydrofuran (20 mL) and methanol (300 mL) gave the objective compound (253) (7.3g, 11.5 mmol).

### Preparation of Compound (113)

In toluene (150 mL) and ethanol (75 mL), dissolved were Compound (253) (7.3 g, 11.5 mmol), Compound (207) (3.3 g, 13.8 mmol) and tetrakispalladium (O) triphenylphosphine (Pd(PPh₃)₄) (1.4 g, 1.2 mmol). To the solution, aqueous 2M sodium carbonate solution (70 mL) was added, and the mixture was stirred at 120° C. under reflux for 5 hours. Then, the mixture was cooled to 25° C., and the reaction was quenched by adding distilled water (200 mL). The reaction mixture was extracted with ethyl acetate (100 mL), and the extract was dried under reduced pressure. Recrystallization from tetrahydrofuran (20 mL) and methanol (300 mL) gave the objective compound (113) (6.0 g, 8.0 mmol, 70%).

¹H NMR(CDCl₃, 200 MHz) δ=1.67(s, 6H), 7.23-7.40(m, 13H), 7.48-7.60(m, 6H), 7.67-7.77(m, 11H),.7.85-7.90(m, 4H)

MS/FAB: 749.3(found) 748.3(calculated)

### Preparation Example 14

### Preparation of Compound (114)

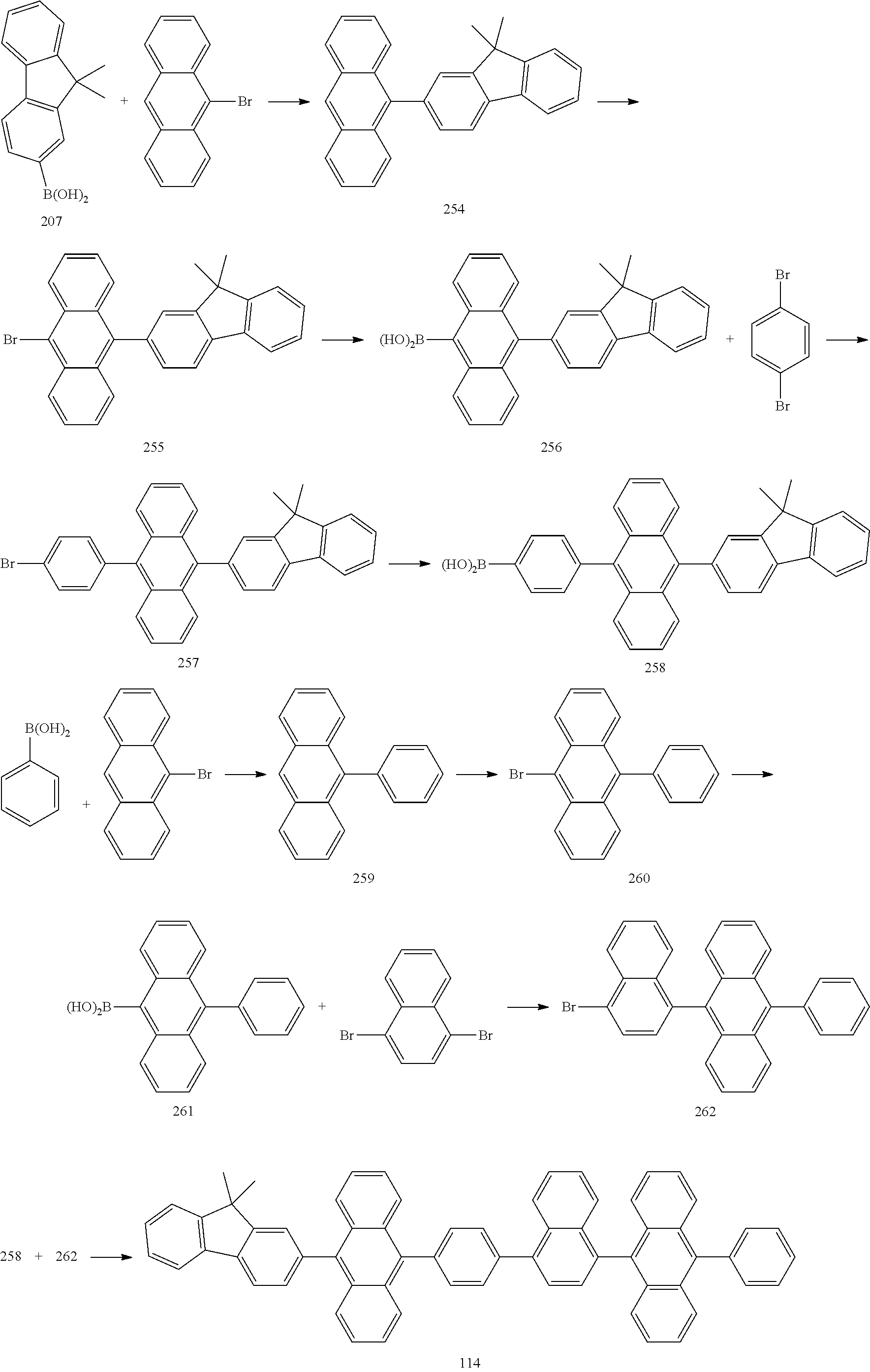

### Preparation of Compound (254)

In toluene (400 mL) and ethanol (200 mL), dissolved were Compound (207) (11.1 g, 46.7 mmol), 9-bromoanthracene (10.0 g, 38.9 mmol) and tetrakispalladium (O) triphenylphosphine (Pd(PPh₃)₄) (4.5 g, 3.9 mmol). To the solution, aqueous 2M sodium carbonate solution (195 mL) was added, and the mixture was stirred at 120° C. under reflux for one day. Then, the mixture was cooled to 25° C., and the reaction was quenched by adding distilled water (500 mL). The reaction mixture was extracted with ethyl acetate (300 mL), and the extract was dried under reduced pressure. Recrystallization from tetrahydrofuran (50 mL) and methanol (500 mL) gave the objective compound (254) (11.5 g, 31.1 mmol).

### Preparation of Compound (255)

Compound (254) (11.5 g, 31.1 mmol) and N-bromosuccinimide (6.6 g, 37.3 mmol) were dissolved in dichloromethane (300 mL) under nitrogen atmosphere, and the solution was stirred at 25° C. for one day. Then, distilled water (400 mL) was added to quench the reaction. The reaction mixture was extracted with dichloromethane (200 mL), and the extract was dried under reduced pressure. Recrystallization from tetrahydrofuran (40 mL) and methanol (500 mL) gave the objective compound (255) (11.2 g, 24.9 mmol).

### Preparation of Compound (256)

Under nitrogen atmosphere, Compound (255) (11.2 g, 24.9 mmol) was dissolved in tetrahydrofuran (200 mL), and the solution was chilled to −78° C. To the solution, slowly added dropwise was n-butyl lithium (n-BuLi, 2.5M in hexane) (12.9 mL, 32.4 mmol). After 30 minutes, trimethylborate (5.7 mL, 49.8 mmol) was added thereto, and the resultant mixture was slowly warmed to room temperature. After stirring at the same temperature for one day, 1N aqueous hydrochloric acid solution (200 mL) was added. The mixture was stirred for 30 minutes, and extracted from water (300 mL) and dichloromethane (200 mL). The extract was dried under reduced pressure and recrystallized from ethyl acetate (30 mL) and hexane (500 mL) to obtain the objective compound (256) (6.2 g, 14.9 mmol).

### Preparation of Compound (257)

In toluene (150 mL) and ethanol (75 mL), dissolved were Compound (256) (6.2 g, 14.9 mmol), 1,4-dibromobenzene (3.2 g, 13.5 mmol) and tetrakispalladium (O) triphenylphosphine (Pd(PPh₃)₄) (1.6 g, 1.4 mmol). To the solution, aqueous 2M sodium carbonate solution (70 mL) was added, and the mixture was stirred at 120° C. under reflux for 2 hours. Then, the mixture was cooled to 25° C., and the reaction was quenched by adding distilled water (200 mL). The reaction mixture was extracted with ethyl acetate (100 mL), and the extract was dried under reduced pressure. Recrystallization from tetrahydrofuran (10 mL) and methanol (100 mL) gave the objective compound (257) (4.3 g, 8.2 mmol).

### Preparation of Compound (258)

Under nitrogen atmosphere, Compound (257) (4.3 g, 8.2 mmol) was dissolved in tetrahydrofuran (100 mL), and the solution was chilled to −78° C. To the solution, slowly added dropwise was n-butyl lithium (n-BuLi, 2.5M in hexane) (4.3 mL, 10.7 mmol). After 30 minutes, trimethylborate (1.9 mL, 16.4 mmol) was added thereto, and the resultant mixture was slowly warmed to room temperature. After stirring at the same temperature for one day, 1N aqueous hydrochloric acid solution (100 mL) was added. The mixture was stirred for 30 minutes, and extracted from water (100 mL) and dichloromethane (100 mL). The extract was dried under reduced pressure and recrystallized from ethyl acetate (20 mL) and hexane (100 mL) to obtain the objective compound (258) (3.2 g, 6.5 mmol).

### Preparation of Compound (259)

In toluene (400 mL) and ethanol (200 mL), dissolved were 9-bromoanthracene (10.0 g, 38.9 mmol), phenylboronic acid (5.7 g, 46.7 mmol) and tetrakispalladium (O) triphenylphosphine (Pd(PPh₃)₄) (4.5 g, 3.9 mmol). To the solution, aqueous 2M sodium carbonate solution (195 mL) was added, and the mixture was stirred at 120° C. under reflux for one day. Then, the mixture was cooled to 25° C., and the reaction was quenched by adding distilled water (500 mL). The reaction mixture was extracted with ethyl acetate (300 mL), and the extract was dried under reduced pressure. Recrystallization from tetrahydrofuran (50 mL) and methanol (500 mL) gave the objective compound (259) (8.9 g, 35.0 mmol).

### Preparation of Compound (260)

Under nitrogen atmosphere, Compound (259) (8.9 g, 35.0 mmol) and N-bromosuccinimide (6.2 g, 42.0 mmol) were dissolved in dichloromethane (200 mL), and the solution was stirred at 25° C. for one day. Then, the reaction was quenched by adding distilled water (300 mL), and the mixture was extracted with dichloromethane (100 mL). The extract was dried under reduced pressure and recrystallized from tetrahydrofuran (20 mL) and methanol (300 mL) to obtain the objective compound (260) (9.9 g, 29.8 mmol).

### Preparation of Compound (261)

Under nitrogen atmosphere, Compound (260) (9.9 g, 29.8 mmol) was dissolved in tetrahydrofuran (200 mL), and the solution was chilled to −78° C. To the solution, slowly added dropwise was n-butyl lithium (n-BuLi, 2.5M in hexane) (15.5 mL, 38.7 mmol). After 30 minutes, trimethylborate (6.8 mL, 59.6 mmol) was added thereto, and the resultant mixture was slowly warmed to room temperature. After stirring at the same temperature for one day, aqueous 1N hydrochloric acid solution (100 mL) was added. The mixture was stirred for 30 minutes, and extracted from water (200 mL) and dichloromethane (200 mL). The extract was dried under reduced pressure and recrystallized from ethyl acetate (20 mL) and hexane (300 mL) to obtain the objective compound (261) (7.1 g, 23.8 mmol).

### Preparation of Compound (262)

In toluene (200 mL) and ethanol (100 mL), dissolved were Compound (261) (7.1 g, 23.8 mmol), 1,4-dibromonaphthalene (7.5 g, 26.2 mmol) and tetrakispalladium (O) triphenylphosphine (Pd(PPh₃)₄) (2.8 g, 2.4 mmol). To the solution, aqueous 2M sodium carbonate solution (120 mL) was added, and the mixture was stirred at 120° C. under reflux for 2 hours. Then, the mixture was cooled to 25° C., and the reaction was quenched by adding distilled water (200 mL). The reaction mixture was extracted with ethyl acetate (100 mL), and the extract was dried under reduced pressure. Recrystallization from tetrahydrofuran (10 mL) and methanol (200 mL) gave the objective compound (262) (6.6 g, 14.4 mmol).

### Preparation of Compound (114)

In toluene (150 mL) and ethanol (75 mL), dissolved were Compound (262) (6.6 g, 14.4 mmol), Compound (258) (8.5 g, 17.3 mmol) and tetrakispalladium (O) triphenylphosphine (Pd(PPh₃)₄) (1.6 g, 1.4 mmol). Aqueous 2M sodium carbonate solution (70 mL) was added to the solution, and the mixture was stirred at 120° C. under reflux for 5 hours. Then, the temperature was lowered to 25° C., and the reaction was quenched by adding distilled water (200 mL). The reaction mixture was extracted with ethyl acetate (100 mL), and the extract was dried under reduced pressure. Recrystallization from tetrahydrofuran (20 mL) and methanol (300 mL) gave the objective compound (114) (7.1 g, 8.6 mmol, 60%).

¹H NMR(CDCl₃, 200 MHz) δ=1.68(s, 6H), 7.23-7.38(m, 15H), 7.48-7.67(m, 20H), 7.78(m, 1H), 7.85-7.90(m, 2H)

MS/FAB: 825.3(found) 824.3(calculated)

### Preparation Example 15

### Preparation of Compound (115)

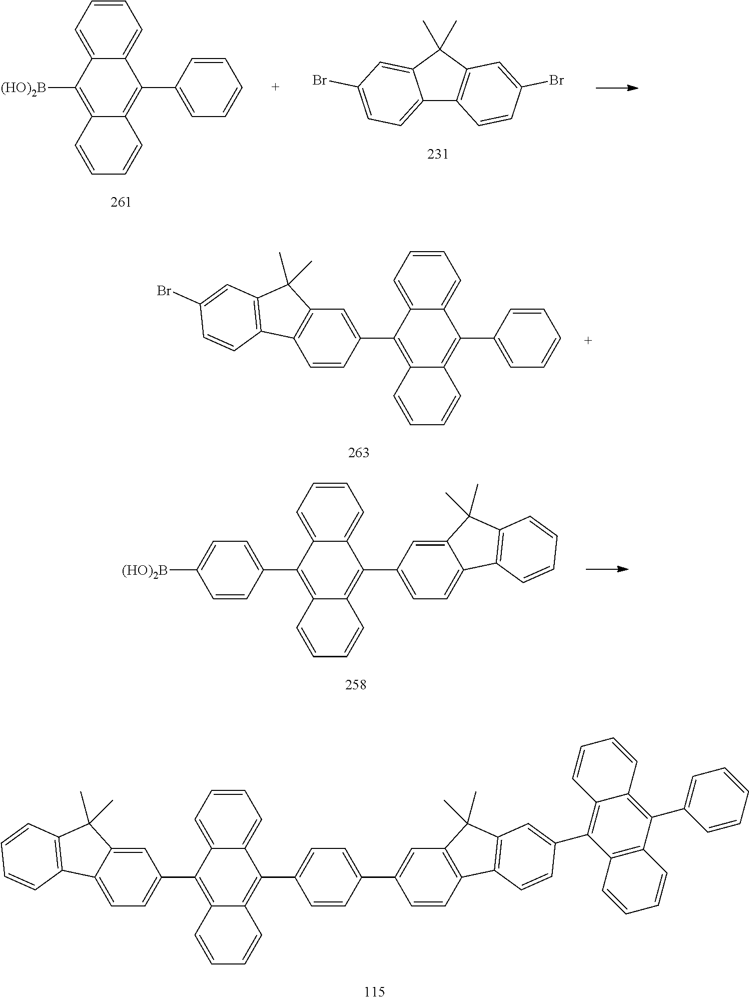

### Preparation of Compound (263)

In toluene (150 mL) and ethanol (75 mL), dissolved were Compound (261) (5.1 g, 17.0 mmol), Compound (231) (5.0 g, 14.2 mmol) and tetrakispalladium (O) triphenylphosphine (Pd(PPh₃)₄) (1.6 g, 1.4 mmol). Aqueous 2M sodium carbonate solution (70 mL) was added to the solution, and the mixture was stirred at 120° C. under reflux for 2 hours. Then, the temperature was lowered to 25° C., and the reaction was quenched by adding distilled water (200 mL). The reaction mixture was extracted with ethyl acetate (100 mL), and the extract was dried under reduced pressure. Recrystallization from tetrahydrofuran (10 mL) and methanol (200 mL) gave the objective compound (263) (4.5 g, 8.6 mmol).

### Preparation of Compound (115)

In toluene (100 mL) and ethanol (50 mL), dissolved were Compound (263) (4.5 g, 8.6 mmol), Compound (258) (5.0 g, 10.3 mmol) and tetrakispalladium (O) triphenylphosphine (Pd(PPh₃)₄) (1.0 g, 0.9 mmol). Aqueous 2M sodium carbonate solution (50 mL) was added to the solution, and the mixture was stirred at 120° C. under reflux for 5 hours. Then, the temperature was lowered to 25° C., and the reaction was quenched by adding distilled water (200 mL). The reaction mixture was extracted with ethyl acetate (100 mL), and the extract was dried under reduced pressure. Recrystallization from tetrahydrofuran (20 mL) and methanol (300 mL) gave the objective compound (115) (3.8 g, 4.3 mmol, 50%).

¹H NMR(CDCl₃, 200 MHz) δ=1.67 (s, 12H), 7.24-7.38 (m, 13H), 7.48-7.67(m, 18H), 7.77(m, 3H), 7.88-7.91(m, 4H)

MS/FAB: 891.3(found) 890.3(calculated)

### Preparation Example 16

### Preparation of Compound (116)

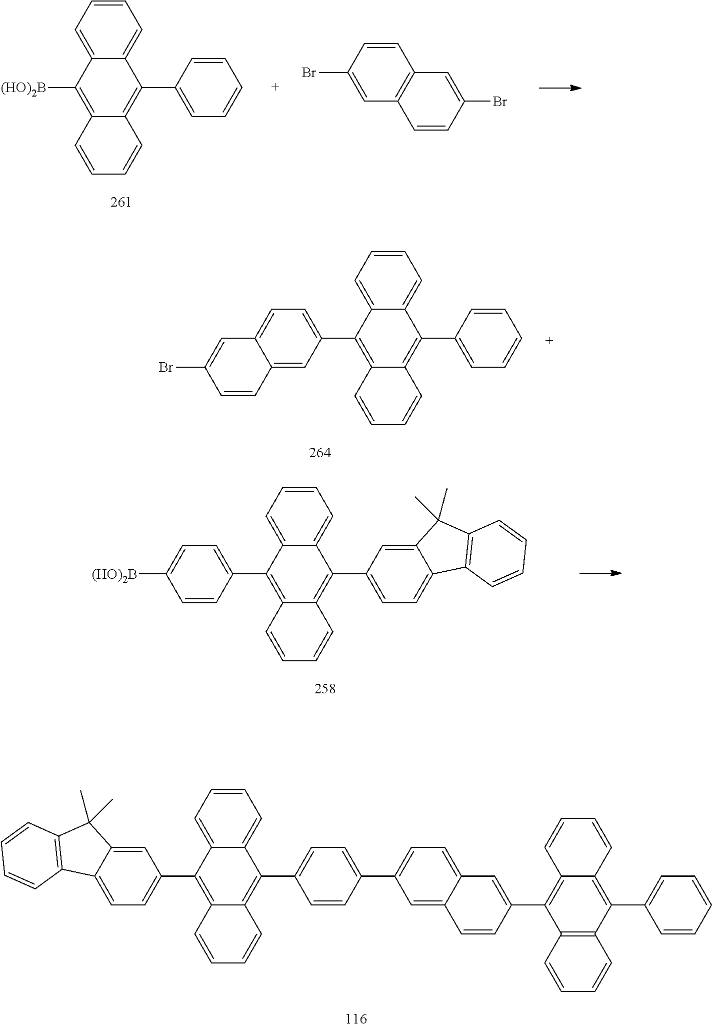

### Preparation of Compound (264)

In toluene (200 mL) and ethanol (100 mL), dissolved were Compound (261) (7.1 g, 23.8 mmol), 2,6-dibromonaphthalene (7.5 g, 26.2 mmol) and tetrakispalladium (O) triphenylphosphine (Pd(PPh₃)₄) (2.8 g, 2.4 mmol). Aqueous 2M sodium carbonate solution (120 mL) was added to the solution, and the mixture was stirred at 120° C. under reflux for 2 hours. Then, the temperature was lowered to 25° C., and the reaction was quenched by adding distilled water (200 mL). The reaction mixture was extracted with ethyl acetate (100 mL), and the extract was dried under reduced pressure. Recrystallization from tetrahydrofuran (10 mL) and methanol (200 mL) gave the objective compound (264) (6.6 g, 14.4 mmol).

### Preparation of Compound (116)

In toluene (150 mL) and ethanol (75 mL), dissolved were Compound (264) (6.6 g, 14.4 mmol), Compound (258) (8.5 g, 17.3 mmol) and tetrakispalladium (O) triphenylphosphine (Pd(PPh₃)₄) (1.6 g, 1.4 mmol). Aqueous 2M sodium carbonate solution (70 mL) was added to the solution, and the mixture was stirred at 120° C. under reflux for 5 hours. Then, the temperature was lowered to 25° C., and the reaction was quenched by adding distilled water (200 mL). The reaction mixture was extracted with ethyl acetate (100 mL), and the extract was dried under reduced pressure. Recrystallization from tetrahydrofuran (20 mL) and methanol (300 mL) gave the objective compound (116) (7.1 g, 8.6 mmol, 60%).

¹H NMR(CDCl₃, 200 MHz) δ=1.68(s, 6H), 7.22-7.38(m, 13H), 7.48-7.60(m, 10H), 7.67-7.73(m, 11H), 7.89-7.90(m, 4H)

MS/FAB: 825.3(found) 824.3(calculated)

### Preparation Example 17

### Preparation of Compound (117)

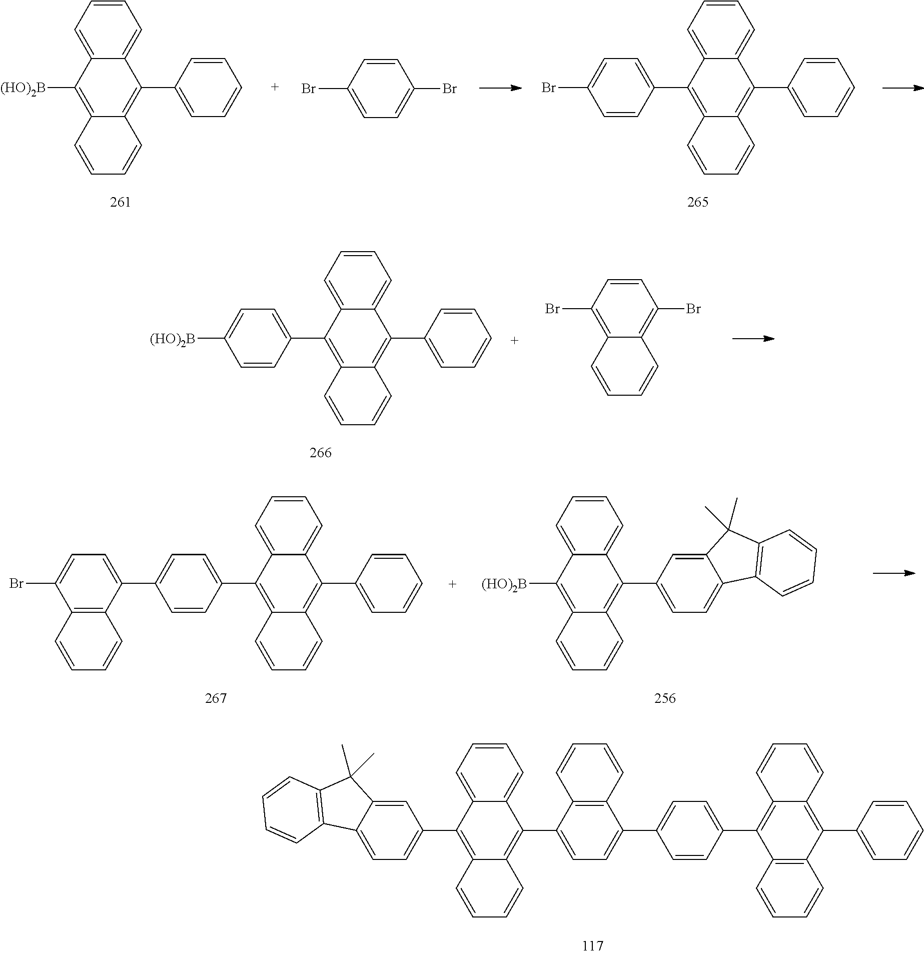

### Preparation of Compound (265)

In toluene (200 mL) and ethanol (100 mL), dissolved were Compound (261) (7.1 g, 23.8 mmol), 1,4-dibromobenzene (6.2 g, 26.2 mmol) and tetrakispalladium (O) triphenylphosphine (Pd(PPh₃)₄) (2.8 g, 2.4 mmol). Aqueous 2M sodium carbonate solution (120 mL) was added to the solution, and the mixture was stirred at 120° C. under reflux for 2 hours. Then, the temperature was lowered to 25° C., and the reaction was quenched by adding distilled water (200 mL). The reaction mixture was extracted with ethyl acetate (100 mL), and the extract was dried under reduced pressure. Recrystallization from tetrahydrofuran (10 mL) and methanol (200 mL) gave the objective compound (265) (5.8 g, 14.3 mmol).

### Preparation of Compound (266)

Under nitrogen atmosphere, Compound (265) (5.8 g, 14.3 mmol) was dissolved in tetrahydrofuran (150 mL), and the solution was chilled to −78° C. To the solution, slowly added dropwise was n-butyl lithium (n-BuLi, 2.5M in hexane) (7.4 mL, 18.6 mmol). After 30 minutes, trimethylborate (3.2 mL, 28.6 mmol) was added thereto, and the resultant mixture was slowly warmed to room temperature. After stirring at the same temperature for one day, aqueous 1N hydrochloric acid solution (100 mL) was added. The mixture was stirred for 30 minutes, and extracted from water (100 mL) and dichloromethane (200 mL). The extract was dried under reduced pressure and recrystallized from ethyl acetate (10 mL) and hexane (200 mL) to obtain the objective compound (266) (2.7 g, 7.2 mmol).

### Preparation of Compound (267)

In toluene (80 mL) and ethanol (40 mL), dissolved were Compound (266) (2.7 g, 7.2 mmol), 1,4-dibromonaphthalene (1.9 6.5 mmol) and tetrakispalladium (O) triphenylphosphine (Pd(PPh₃)₄) (0.9 g, 0.8 mmol). To the solution, aqueous 2M sodium carbonate solution (40 mL) was added, and the mixture was stirred at 120° C. under reflux for 2 hours. Then, the mixture was cooled to 25° C., and the reaction was quenched by adding distilled water (100 mL). The reaction mixture was extracted with ethyl acetate (50 mL), and the extract was dried under reduced pressure. Recrystallization from tetrahydrofuran (5 mL) and methanol (100 mL) gave the objective compound (267) (2.3 g, 4.3 mmol).

### Preparation of Compound (117)

In toluene (50 mL) and ethanol (25 mL), dissolved were Compound (267) (2.3 g, 4.3 mmol), Compound (256) (2.1 g, 5.2 mmol) and tetrakispalladium (O) triphenylphosphine (Pd(PPh₃)₄) (0.5 g, 0.4 mmol). Aqueous 2M sodium carbonate solution (25 mL) was added to the solution, and the mixture was stirred at 120° C. under reflux for 5 hours. Then, the temperature was lowered to 25° C., and the reaction was quenched by adding distilled water (100 mL). The reaction mixture was extracted with ethyl acetate (50 mL), and the extract was dried under reduced pressure. Recrystallization from tetrahydrofuran (5 mL) and methanol (100 mL) gave the objective compound (117) (2.1 g, 2.5 mmol, 60%).

¹H NMR(CDCl₃, 200 MHz) δ=1.67(s, 6H), 7.23-7.38(m, 15H), 7.48-7.55(m, 7H), 7.60-7.67(m, 13H), 7.78(m, 1H), 7.85-7.91(m, 2H)

MS/FAB: 825.3(found) 824.3(calculated)

### Preparation Example 18

### Preparation of Compound (118)

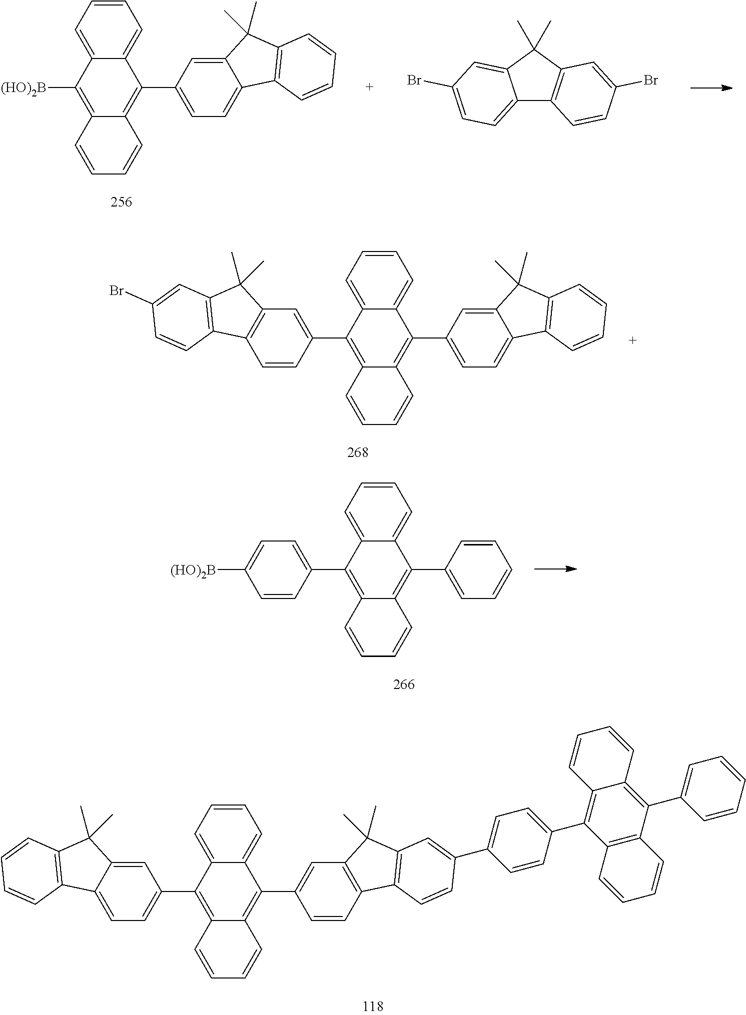

### Preparation of Compound (268)

In toluene (150 mL) and ethanol (75 mL), dissolved were Compound (256) (6.5 g, 15.6 mmol), 2,7-dibromofluorene (5.0 g, 14.2 mmol) and tetrakispalladium (O) triphenylphosphine (Pd(PPh₃)₄) (1.6 g, 1.4 mmol). To the solution, aqueous 2M sodium carbonate solution (71 mL) was added, and the mixture was stirred at 120° C. under reflux for 2 hours. Then, the mixture was cooled to 25° C., and the reaction was quenched by adding distilled water (200 mL). The reaction mixture was extracted with ethyl acetate (100 mL), and the extract was dried under reduced pressure. Recrystallization from tetrahydrofuran (10 mL) and methanol (200 mL) gave the objective compound (268) (5.9 g, 9.2 mmol).

### Preparation of Compound (118)

In toluene (100 mL) and ethanol (50 mL), dissolved were Compound (268) (5.9 g, 9.2 mmol), Compound (266) (4.1 g, 11.0 mmol) and tetrakispalladium (O) triphenylphosphine (Pd(PPh₃)₄) (1.0 g, 0.9 mmol). Aqueous 2M sodium carbonate solution (50 mL) was added to the solution, and the mixture was stirred at 120° C. under reflux for 5 hours. Then, the temperature was lowered to 25° C., and the reaction was quenched by adding distilled water (150 mL). The reaction mixture was extracted with ethyl acetate (100 mL), and the extract was dried under reduced pressure. Recrystallization from tetrahydrofuran (10 mL) and methanol (200 mL) gave the objective compound (118) (3.3 g, 3.7 mmol, 45%).

¹H NMR(CDCl₃, 200 MHz) δ=1.68 (s, 12H), 7.23-7.40 (m, 13H), 7.50-7.67(m, 18H), 7.78(m, 3H), 7.86-7.91(m, 4H)

MS/FAB: 891.3(found) 890.3(calculated)

### Preparation Example 19

### Preparation of Compound (119)

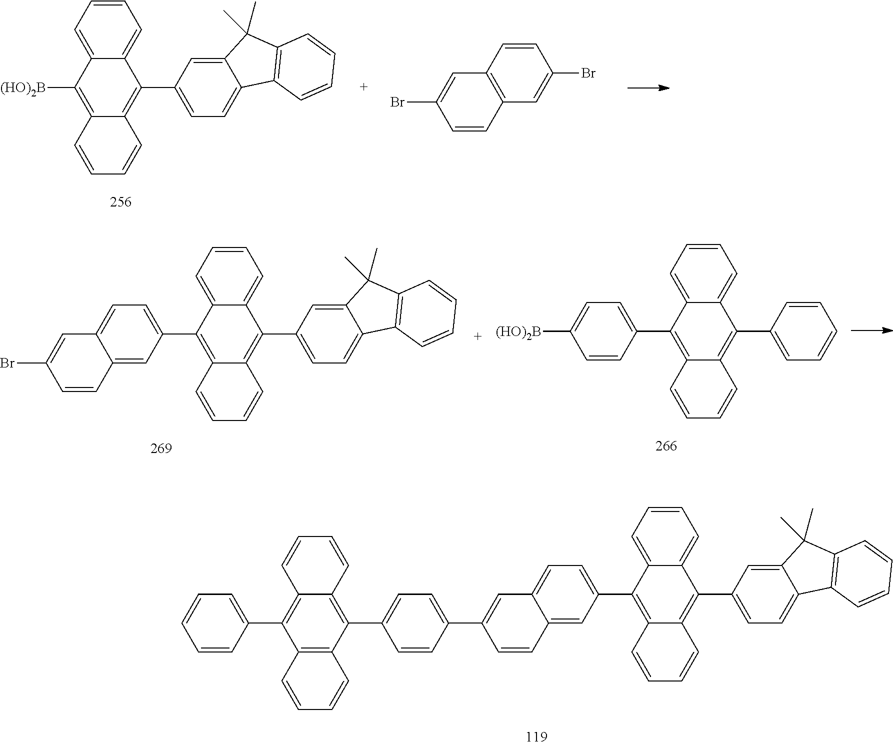

### Preparation of Compound (269)

In toluene (200 mL) and ethanol (100 mL), dissolved were Compound (256) (6.5 g, 19.2 mmol), 2,6-dibromonaphthalene (5.0 g, 17.5 mmol) and tetrakispalladium (O) triphenylphosphine (Pd(PPh₃)₄) (2.1 g, 1.8 mmol). To the solution, aqueous 2M sodium carbonate solution (90 mL) was added, and the mixture was stirred at 120° C. under reflux for 2 hours. Then, the mixture was cooled to 25° C., and the reaction was quenched by adding distilled water (300 mL). The reaction mixture was extracted with ethyl acetate (200 mL), and the extract was dried under reduced pressure. Recrystallization from tetrahydrofuran (20 mL) and methanol (300 mL) gave the objective compound (269) (6.0 g, 10.5 mmol).

### Preparation of Compound (119)

In toluene (100 mL) and ethanol (50 mL), dissolved were Compound (269) (6.0 g, 10.5 mmol), Compound (266) (4.7 g, 12.6 mmol) and tetrakispalladium (O) triphenylphosphine (Pd(PPh₃)₄) (1.3 g, 1.1 mmol). Aqueous 2M sodium carbonate solution (50 mL) was added to the solution, and the mixture was stirred at 120° C. under reflux for 5 hours. Then, the temperature was lowered to 25° C., and the reaction was quenched by adding distilled water (150 mL). The reaction mixture was extracted with ethyl acetate (100 mL), and the extract was dried under reduced pressure. Recrystallization from tetrahydrofuran (10 mL) and methanol (200 mL) gave the objective compound (119) (6.5 g, 7.9 mmol, 75%).

¹H NMR (CDCl₃, 200 MHz) δ=1.67 (s, 6H), 7.24-7.38(m, 13H), 7.48-7.55(m, 10H), 7.67-7.77(m, 11H), 7.87-7.90(m, 4H)

MS/FAB: 825.3(found) 824.3(calculated)

### Example 1

### Manufacture of an OLED using the Compound According to the Present Invention

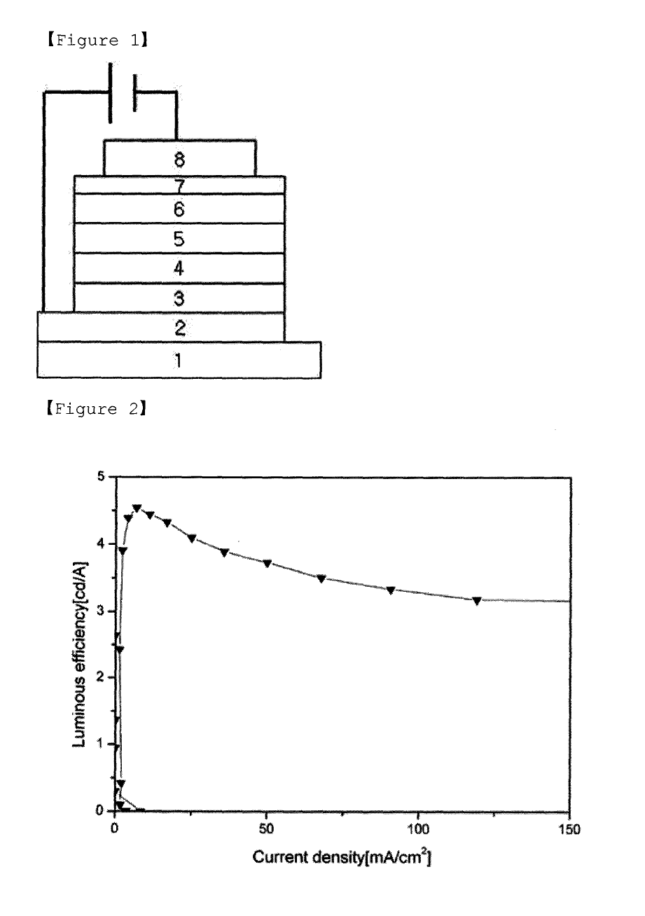

An OLED was manufactured as shown in FIG. 1 by employing an EL material according to the present invention as a host material.

First, a transparent electrode ITO thin film (15Ω/□) (2) obtained from glass for OLED (1) (manufactured by Samsung Corning) was subjected to ultrasonic washing with trichloroethylene, acetone, ethanol and distilled water, subsequently, and stored in isopronanol before use.

Then, an ITO substrate was equipped in a substrate folder of a vacuum vapor-deposit device, and 4,4′,4″-tris(N,N-(2-naphthyl)-phenylamino)triphenylamine (2-TNATA) was placed in a cell of the vacuum vapor-deposit device, which was then vented to reach 10⁻⁶ torr of vacuum in the chamber. Electric current was applied to the cell to evaporate 2-TNATA to vapor-deposit a hole injection layer (3) with 60 nm of thickness on the ITO substrate.

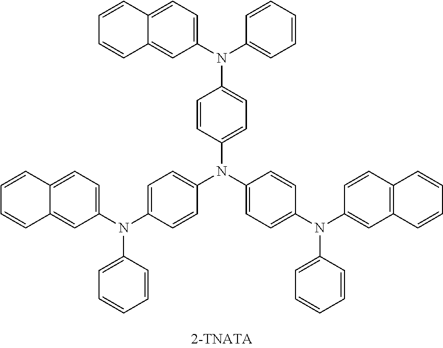

Then, another cell of the vacuum vapor-deposit device was charged with N,N′-bis(a-naphthyl)-N,N′-diphenyl-4,4′-diamine (NPB) (having the structural formula shown below), and electric current was applied to the cell to evaporate NPB to vapor-deposit a hole transportation layer (4) with 20 nm of thickness on the hole injection layer.

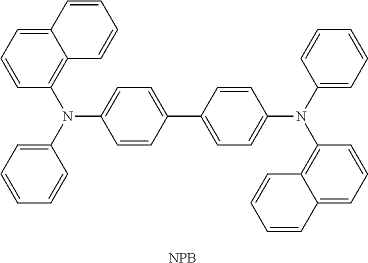

After formation of the hole injection layer and the hole transportation layer, an EL layer was vapor-deposited as follows. One cell of the vacuum deposition device was charged with an EL compound according to the present invention (e.g. Compound 105), while another cell of said device with perylene (having the structural formula shown below). Both cells were heated to vapor-deposite perylene with the vapor-deposition rate of 2˜5 mol% to result in an electroluminescent layer (5) with a thickness of 30 nm on the hole transportation layer.

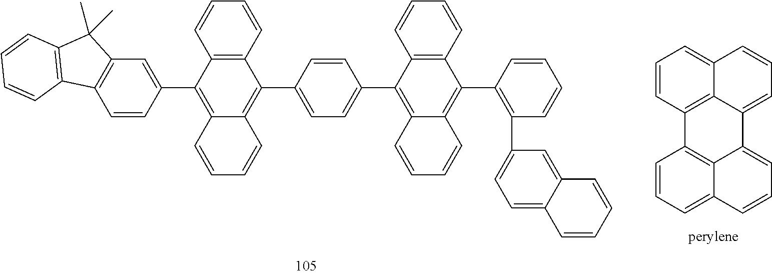

Then, tris(8-hydroxyquinoline)-aluminum (III) (Alq) (of which the structure is shown below) was vapor-deposited with a thickness of 20 nm, as an electron transportation layer (6), followed by lithium quinolate (Liq) with a thickness of 1 to 2 nm as an electron injection layer (7). Thereafter, an Al cathode (8) was vapor-deposited with a thickness of 150 nm by using another vacuum vapor-deposit device to manufacture an OLED.

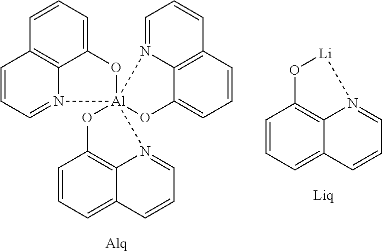

Individual materials used for the OLED's were purified by vacuum sublimation under 10⁻⁶ torr to be employed as an EL material for the OLED's.

### Comparative Example 1

### Manufacture of an OLED using Conventional EL Material

A hole injection layer (3) and a hole transportation layer (4) were formed according to the same procedure as described in Example 1, and dinaphthylanthracene (DNA) as a blue electroluminescent material was charged in one cell of said vapor-deposit device, while in another cell perylene as another blue electroluminescent material. Then, an electroluminescent layer (5) with 30 nm of thickness was vapor-deposited on said hole transportation layer with vapor-deposit rate of 100:1.

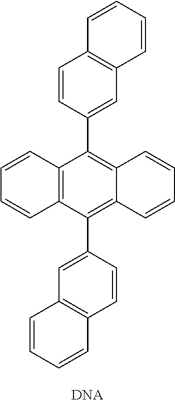

Then, an electron transportation layer (6) and an electron injection layer (7) were vapor-deposited according to the same procedure as described in Example 1, and an Al cathode (8) was vapor-deposited by using another vacuum vapor-deposit device with a thickness of 150 nm, to manufacture an OLED.

### Example 2

### Electroluminescent Properties of the OLED Manufactured

Luminous efficiencies of OLED's comprising the organic electroluminescent compound according to the present invention prepared from Example 1 and the conventional electroluminescent compound from Comparative Example 1 were measured at 500 cd/m² and 2,000 cd/m², respectively, of which the results are shown in Table 1. Since the luminescent properties in the range of low luminance and those applied on the panel are very important in case of a blue electroluminescent material, in particular, the data of luminance of about 2,000 cd/m² were established as the standard in order to reflect those properties.

TABLE 1

| | | | | Luminous | | | |
|---|---|---|---|---|---|---|---|
| | | | Doping | efficiency | | | |
| | | | concen- | (cd/A) | | Color | |
| | | | tration | @500 | @2,000 | coordinate | |
| No. | Host | Dopant | (mol %) | cd/m² | cd/m² | X | Y |
| 1 | 101 | perylene | 2.0 | 5.61 | 5.21 | 0.15 | 0.20 |
| 2 | 102 | perylene | 3.0 | 6.17 | 5.86 | 0.15 | 0.18 |
| 3 | 103 | perylene | 2.0 | 5.28 | 5.05 | 0.15 | 0.18 |
| 4 | 104 | perylene | 2.0 | 6.04 | 5.89 | 0.15 | 0.19 |
| 5 | 105 | perylene | 2.0 | 6.57 | 6.12 | 0.15 | 0.20 |
| 6 | 106 | perylene | 2.0 | 5.65 | 5.35 | 0.16 | 0.20 |
| 7 | 107 | perylene | 5.0 | 6.33 | 6.07 | 0.16 | 0.19 |
| 8 | 108 | perylene | 2.0 | 5.37 | 5.10 | 0.15 | 0.18 |
| 9 | 109 | perylene | 3.0 | 5.64 | 5.19 | 0.16 | 0.20 |
| 10 | 110 | perylene | 2.0 | 6.22 | 5.89 | 0.15 | 0.19 |
| 11 | 111 | perylene | 2.0 | 5.76 | 5.28 | 0.15 | 0.19 |
| 12 | 112 | perylene | 3.0 | 5.53 | 5.16 | 0.15 | 0.18 |
| 13 | 113 | perylene | 2.0 | 5.60 | 5.19 | 0.15 | 0.19 |
| 14 | 114 | perylene | 2.0 | 5.88 | 5.40 | 0.15 | 0.18 |
| 15 | 115 | perylene | 3.0 | 5.49 | 4.88 | 0.15 | 0.19 |
| 16 | 116 | perylene | 2.0 | 5.77 | 5.36 | 0.15 | 0.18 |
| 17 | 117 | perylene | 2.0 | 5.49 | 4.96 | 0.16 | 0.19 |
| 18 | 118 | perylene | 3.0 | 5.87 | 5.35 | 0.15 | 0.18 |
| 19 | 119 | perylene | 2.0 | 5.72 | 5.26 | 0.15 | 0.19 |
| Comp. 1 | DNA | perylene | 2.0 | 4.45 | 3.62 | 0.16 | 0.20 |

Table 1 shows the results obtained by applying the material of the present invention to a blue OLED. As can be seen from Table 1, the efficiency of the EL material according to the present invention was superior by at least 1 cd/A when compared to that of Comparative Example 1. Particularly, in high luminance, improvement by not less than 1.5 cd/A was found depending on the compounds.

With respect to the color purity, slight improvement was observed when the host material according to the present invention was applied. Improvements in both color purity and luminous efficiency verify excellent features of the EL materials according to the present invention.

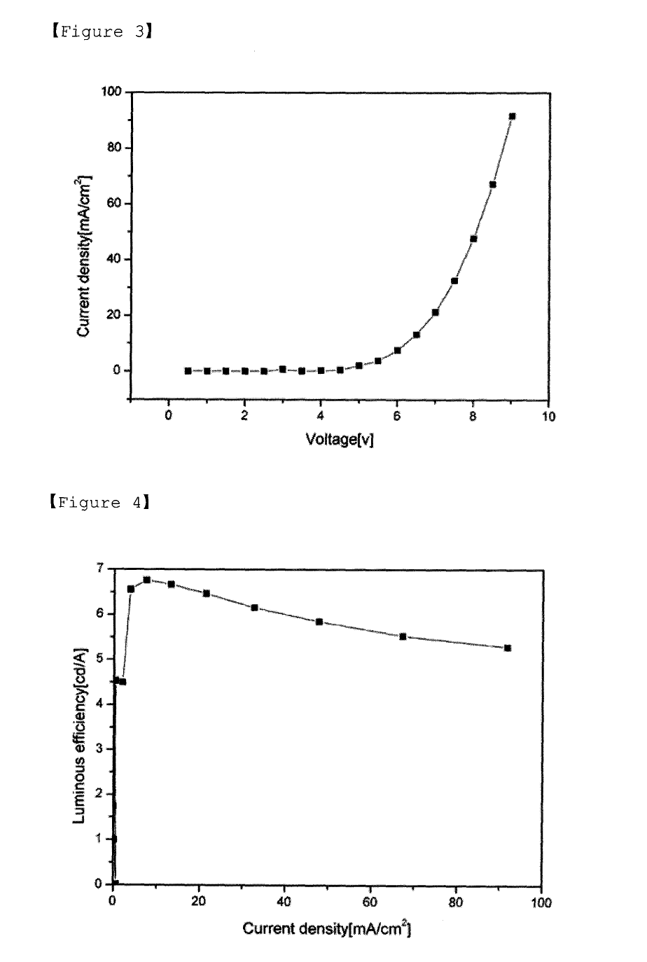

FIG. 2 is a luminous efficiency-current density curve of Comparative Example 1, which employs DNA:perylene as conventional EL material. FIG. 3 and FIG. 4 are current density-voltage and luminous efficiency-current density curves, respectively, of a blue OLED which employs Compound (105) according to the present invention as EL material. As can be seen from the drawings, noticeable improvement of properties was confirmed.

It is verified, as described above, that such improvements in performances of EL materials could be realized by simultaneously applying the concept of connecting two anthracenes via a backbone having a role of a spacer and the concept of comprising asymmetric branches.

### INDUSTRIAL APPLICABILITY

The organic EL compounds according to the present invention provide good luminous efficiency and excellent life property, and thus enable to manufacture OLED devices with very good operation lifetime.

1. An organic electroluminescent compound represented by Chemical Formula (1): 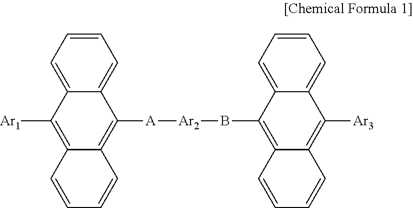
wherein, A and B independently represent a chemical bond or C₆-C₃₀ arylene with or without one or more substituent(s) selected from C₁-C₂₀ alkyl, C₁-C₂₀ alkoxy, C₆-C₃₀ aryl, C₄-C₃₀ heteroaryl and halogen(s), provided that A and B are not fluorenylene;
Ar₁ is an aryl group selected from the following structures, and the aryl of Ar₁ may have one substituent selected from C₁-C₂₀ alkyl, C₁-C₂₀ alkoxy, C₆-C₃₀ aryl and C₄-C₃₀ heteroaryl; 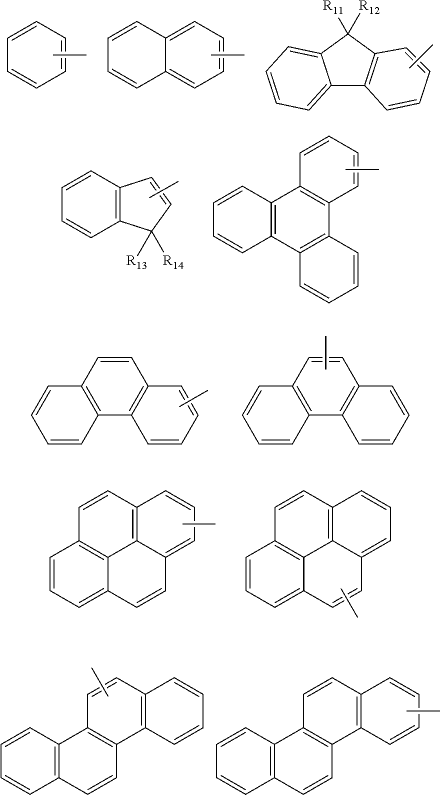
Ar₂ represents phenylene, naphthylene, anthrylene, fluorenylene, phenanthrylene, tetracenylene, naphthacenylene, chrysenylene, pentacenylene, pyrenylene, or the chemical group having the following structure; provided that Ar₂ is not fluorenylene if both A and B are chemical bonds; and the arylene of Ar₂ may have one or more substituent(s) selected from C₁-C₂₀ alkyl, C₁-C₂₀ alkoxy, C₆-C₃₀ aryl, C₄-C₃₀ heteroaryl and a halogen; 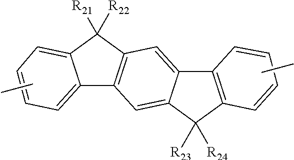
Ar₃ is an aryl group selected from the following structures, and the aryl group of Ar₃ may have one or two substituent(s) selected from C₁-C₂₀ alkyl, C₁-C₂₀ alkoxy, C₆-C₃₀ aryl and C₄-C₃₀ heteroaryl; 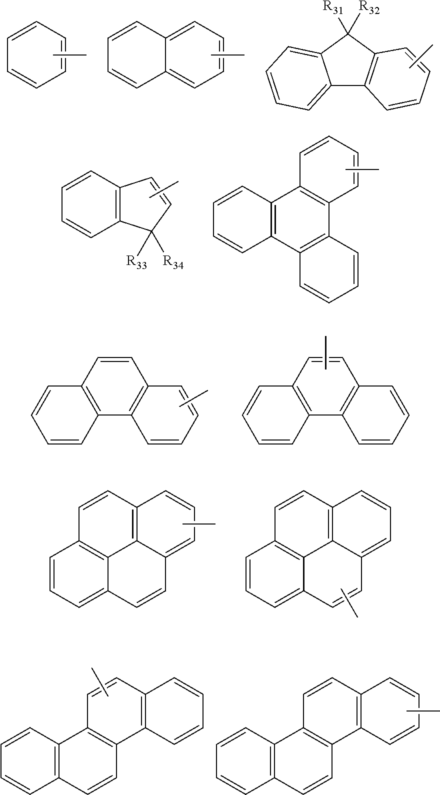
R₁₁, R₁₂, R₁₃ and R₁₄ independently represent hydrogen, C₁-C₂₀ alkyl or C₆-C₃₀ aryl;
R₂₁, R₂₂, R₂₃ and R₂₄ independently represent hydrogen, C₁-C₂₀ alkyl, C₁-C₂₀ alkoxy, C₆-C₃₀ aryl, C₄-C₃₀ heteroaryl or a halogen; and
R₃₁, R₃₂, R₃₃ and R₃₄ independently represent hydrogen, C₁-C₂₀ alkyl or C₆-C₃₀ aryl.

2. An organic electroluminescent compound according to claim 1, wherein Ar₂ is selected from the following structures: 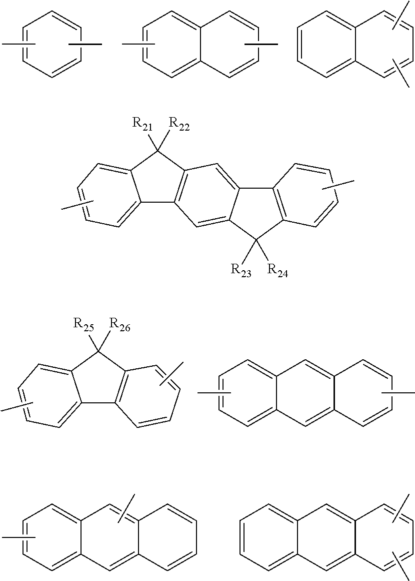
wherein, R₂₁, R₂₂, R₂₃, R₂₄, R₂₅ and R₂₆ independently represent hydrogen, C₁-C₂₀ alkyl, C₁-C₂₀ alkoxy, C₆-C₃₀ aryl, C₄-C₃₀ heteroaryl or a halogen.

3. An organic electroluminescent compound according to claim 2, wherein -A-Ar₂—B— is selected from the following structures: 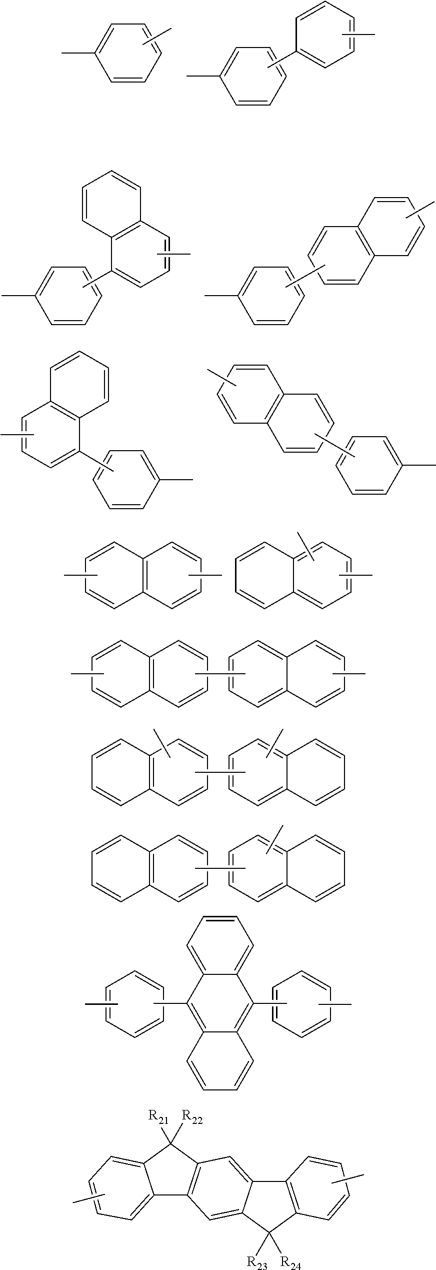 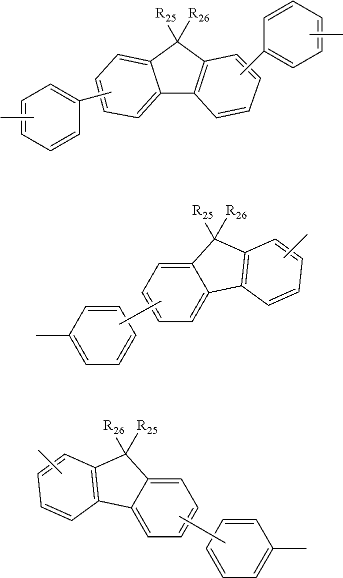
wherein, R₂₁, R₂₂, R₂₃, R₂₄, R₂₅ and R₂₆ independently represent hydrogen, methyl, ethyl, propyl, isopropyl, butyl, isobutyl, pentyl, hexyl, ethylhexyl, heptyl, octyl, isooctyl, nonyl, decyl, dodecyl, hexadecyl, phenyl, tolyl, biphenyl, benzyl, naphthyl, anthryl or fluorenyl.

4. An organic electroluminescent compounds according to claim 3, which is selected from the following compounds:

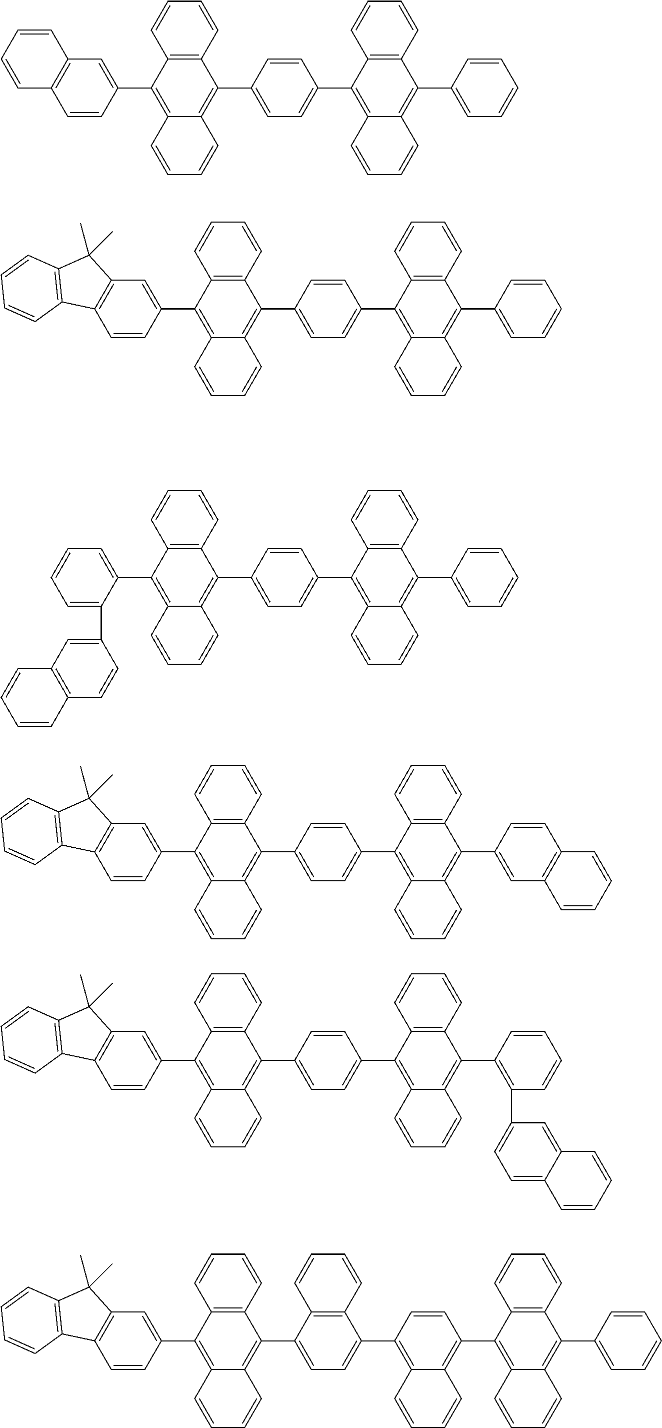

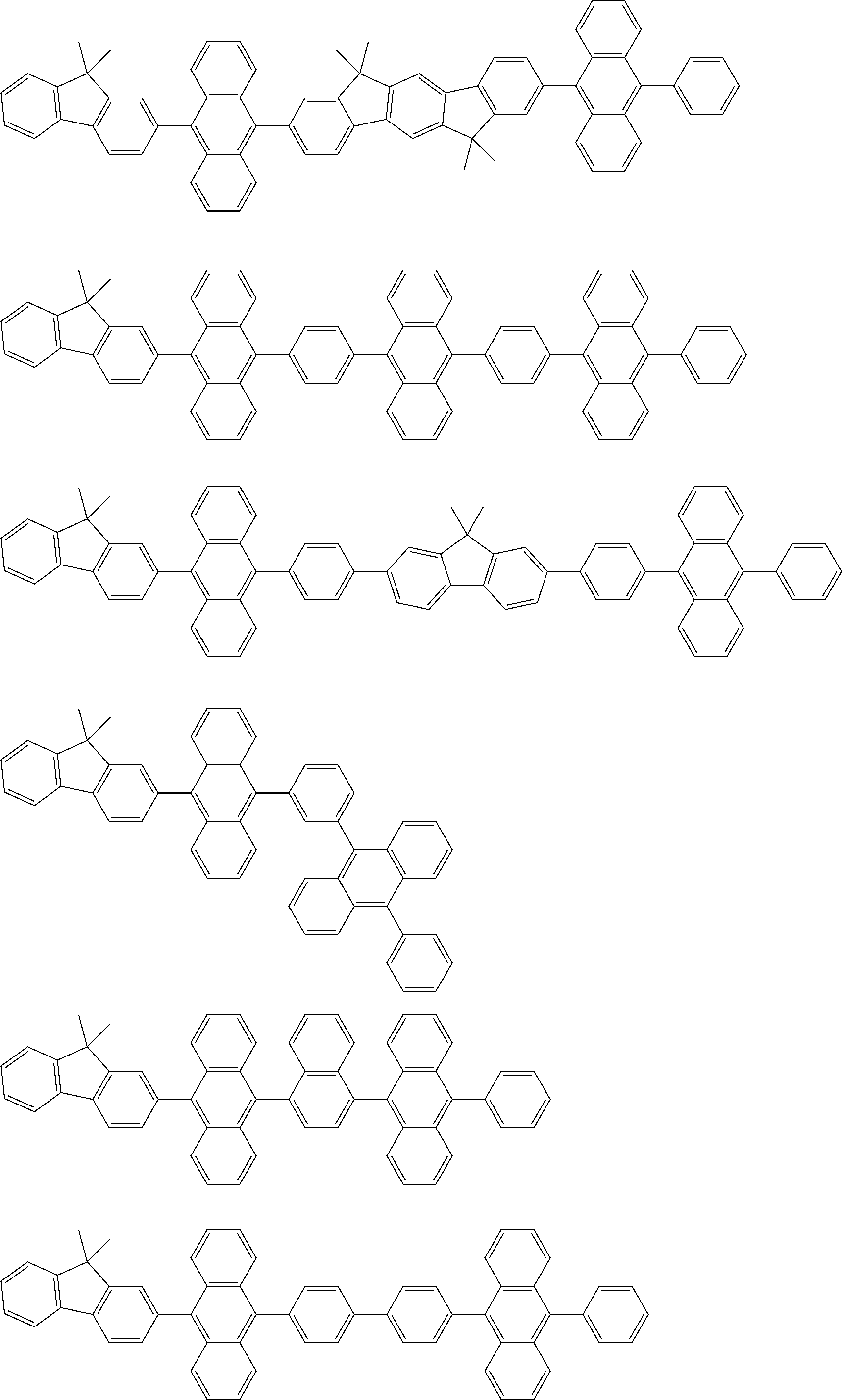

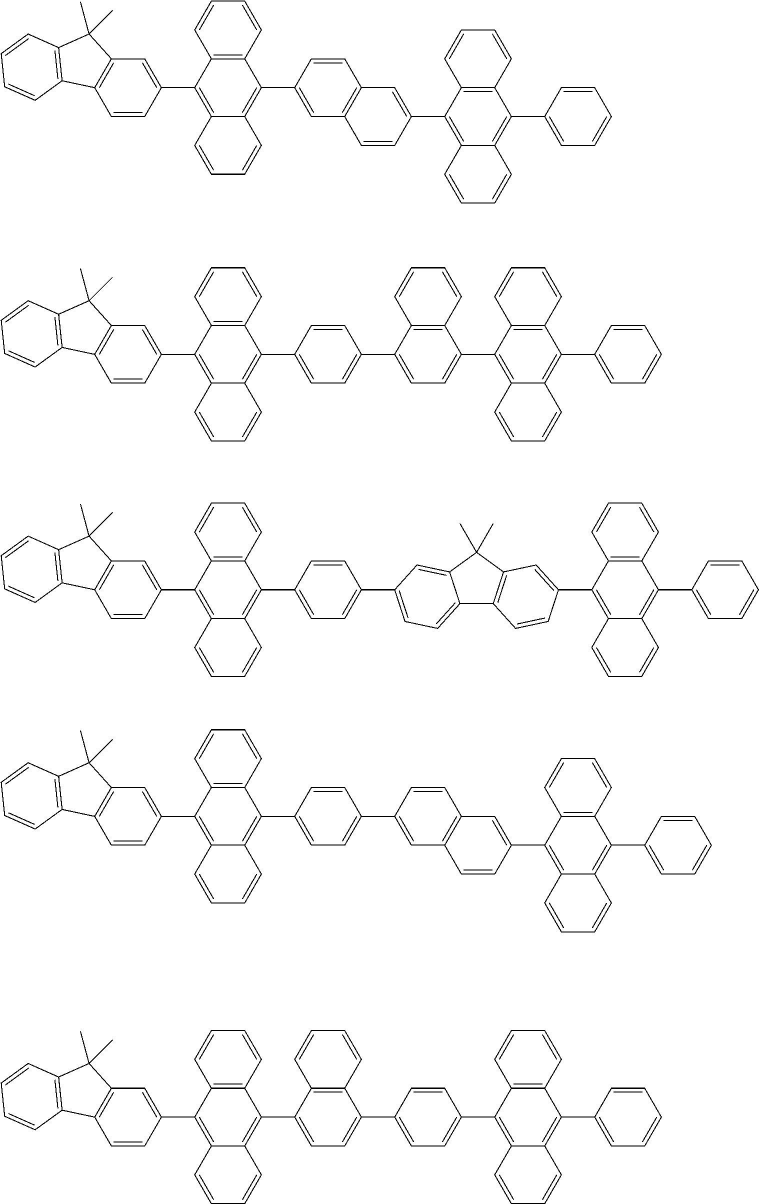

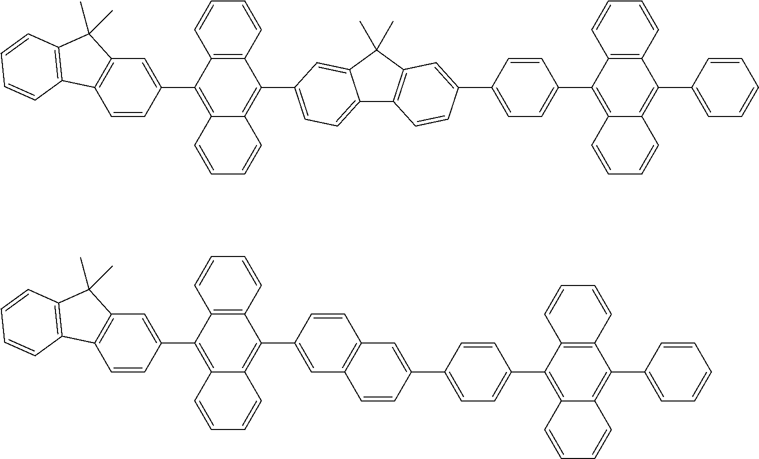

5. An organic light-emitting diode which comprises an organic electroluminescent compound according to any one of claims 1 to 4.
